SEQUENCE LISTING (8 sequences)

SEQ ID NO: 1 - protein, 413 aa, Artificial Sequence
  Description of Artificial Sequence Calcineurin modulator
XXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXKQFLISPPASPPVXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXX

SEQ ID NO: 2 - protein, 13 aa, Artificial Sequence
  Description of Artificial Sequence Calcineurin modulator
KQFLISPPASPPV

SEQ ID NO: 3 - protein, 10 aa, Artificial Sequence
  Description of Artificial Sequence HIV TAT 10mer peptide
GRKKRRQRRR

SEQ ID NO: 4 - protein, 25 aa, Artificial Sequence
  Description of Artificial Sequence Adapt78 peptide inhibitor of calcineurin conjugated to the HIV TAT protein conjugated to a  Calcineurin modulator
GRKKRRQRRRPPKQFLISPPASPPV

SEQ ID NO: 5 - protein, 12 aa, Artificial Sequence
  Description of Artificial Sequence HIV TAT protein transduction domain followed by two glycine residues
GRKKRRQRRRGG

SEQ ID NO: 6 - protein, 12 aa, Artificial Sequence
  Description of Artificial Sequence Adapt78 epitope
GRKKRRQRRRGG

SEQ ID NO: 7 - protein, 113 aa, Artificial Sequence
  Description of Artificial Sequence Calcineurin modulator
XXXXXXXXXXXXXXXXXXXXXXXXXXXXXXXXXXXXXXXXXXXXXXXXXXXXFLISXXASXXVXXXXXXXXXXXXXXXXXXXXXXXXXXXXXXXXXXXXXXXXXXXXXXXXXX

SEQ ID NO: 8 - protein, 32 aa, Artificial Sequence
  Description of Artificial Sequence Calcineurin modulator
PDKQFLISPPASPPVGWKQVPKPKIIQTRRPE

(19) United States
(12) Patent Application Publication
(10) Pub. No.: US 2003/0045679 A1
(43) Pub. Date: Mar. 6, 2003

(54) Calcineurin modulators

(76) Inventor: Dana R. Crawford, Delmar, NY (US)

Correspondence Address: Ivor R. Elrifi, MINTZ, LEVIN, COHN, FERRIS,, GLOVSKY and POPEO, P.C., One Financial Center, Boston, MA, 02111, US

(21) Appl. No.: 10/194,155

(22) Filed: Jul. 12, 2002

Related U.S. Application Data
(60) Provisional application No. 60/305,202, filed on Jul. 13, 2001.

(51) Int. Cl. C07K 14/435; C07K 7/08; C07K 7/06

(52) U.S. Cl. 530/324; 530/325; 530/327; 530/328; 530/326

(57) ABSTRACT

This invention relates to compositions which are useful for inhibiting and potentiating the activity of cellular calcineurin. These compositions include linear peptides, cyclic peptides, peptide analogs, peptidomimetics, combinatorial chemicals, and whole proteins. The compositions can be used to treat calcineurin- and adapt78-related pathologies such as cardiac, brain, immune system and developmental abnormalities; to protect human cells and tissues against stress damage based on the cytoprotective activity of adapt78; and to modulate cell growth based on the growth-altering activity of adapt78. Transgenic animals overexpressing the human adapt78 transgene are also disclosed, e.g., for developing therapeutic treatments against adapt78- and calcineurin-related pathologies and abnormalities such as cardiac hypertrophy, memory loss, immune system dysfunction, developmental disabilities.

### RELATED APPLICATIONS

This application claims the benefit of priority under 35 U.S.C. 119(e) to copending U.S. Provisional Application No. 60/305,202, filed on Jul. 13, 2001, the entire contents of which are incorporated herein by reference.

### FIELD OF THE INVENTION

This invention relates to compositions that are useful for inhibiting and potentiating cellular calcineurin activity. More particularly the invention relates to calcineurin-inhibiting polypeptides, and polynucleotides encoding them, as well as vectors, host cells, antibodies and recombinant methods for producing the polypeptides and polynucleotides.

### BACKGROUND OF THE INVENTION

Calcineurin is a protein phosphatase involved in cellular response to calcium. It mediates a wide range of cellular functions in mammals including T-cell activation, apoptosis of T-lymphocytes and neuronal cells, regulation of NMDA receptor channels, promotion of neurite outgrowth, long-term memory and long term potentiation, skeletal and cardiac muscle growth and differentiation, cytokine gene synthesis, and regulation of neurotransmitter release in brain where it comprises over 1% of the total protein. Thus, calcineurin is an important regulator of many physiological systems in the body.

Calcineurin is activated by elevated calcium in the cytosol followed by calcium and calmodulin binding. It is structurally and functionally conserved from yeast to man, and is a heterodimer consisting of a catalytic A and regulatory B subunits. Its biological role has been further advanced by the discovery that calcineurin is the target of the immunosuppressive drugs FK506 and cyclosporin. FK506 and cyclosporin, in association with FKBP12 and cyclophilin A, respectively, inhibit calcineurin. Gorlach et al. (EMBO J 19:3618-3629 (2000)) demonstrated that calcineurin binding to CBP1 (the Adapt78 homolog in C. neoformans) is inhibited by FK506-FKBP12. The regulation of NF-AT is an important activity of calcineurin. NF-AT comprises a family of four transcriptional regulatory proteins (NF-AT1, 2, 3 and 4) that transduce cytoplasmic signals to the nucleus, where they help regulate selected gene transcription. Overall, calcineurin activation by elevated calcium results in phosphate group removal from members of the NF-AT family via calcineurin phosphatase activity. Dephosphorylated NF-AT proteins then migrate from the cytoplasm to the nucleus where they bind to target genes to promote transcription. The most studied calcineurin-NF-AT signal pathway has been in lymphocytes, where elevation of calcium by cell surface binding of antigen activates calcineurin, ultimately leading to NF-AT-mediated induction of genes involved in T-cell activation such as interleukin-2. NF-AT also appears to be an important mediator of activated calcineurin in both muscle and brain, including cardiac hypertrophy.

Based on these pleitrophic calcineurin actions, aberrant expression of calcineurin would appear to be associated with a wide range of pathological conditions. Such a link has already been demonstrated by a number of studies. Transgenic calcineurin overexpression recapitulates both familial and some acquired forms of cardiac hypertrophy and heart failure; this hypertrophy is inhibited by the classic calcineurin inhibitors cyclosporin A and FK506. Knockout mice to specific calcineurin isoforms have been shown to be defective in in vivo antigen-specific T-cell responses; to accumulate hyperphosphorylated tau protein and exhibit cytoskeletal changes in the hippocampus; and to abolish synaptic depotentiation, important in memory and learning. Stimulation of calcineurin phosphatase activity by calcium was found to be responsible for apoptosis in mammalian cells deprived of growth factors.

Calcineurin inhibitors therefore hold potential clinical benefit in treating calcineurin-associated pathologies, including immune system, brain, skeletal and cardiac muscle dysfunction, and cancer (e.g., via calcineurin's role in apoptosis). Known calcineurin inhibitors i.e., cyclosporin A and FK506, have toxic side effects such as nephrotoxicty, hepatotoxicity, hypertension, central nervous system disturbances, immune suppression (when not used specifically for this purpose), etc. Thus, new calcineurin inhibitors with less toxic side effects would be of significant clinical benefit.

### SUMMARY OF THE INVENTION

The present invention relates to the discovery that calcineurin activity may be modulated with a calcineurin modulator of the invention. The calcineurin modulators of the invention include: an active portion or fragment of Adapt78 protein (or a family member or homolog thereof, such as CBP1); a modified full-length Adapt78 protein or family member or homolog thereof; as well as linear peptides, cyclic peptides, peptide analogs, peptidomimetics, mimics, combinatorial chemicals, and whole proteins. The calcineurin modulator can be administered in a pharmaceutically acceptable form for the treatment of calcineurin-associated pathologies, such as immune system dysfunction, cardiac hypertrophy, Alzheimer's disease and cancer. The calcineurin modulators of the invention, i.e., in the form of a pharmaceutical composition, have less toxic side effects than those presently in use for the treatment of calcineurin-associated or Adapt78-associated pathologies.

The invention also includes isolated nucleic acid molecules including, e.g., nucleic acid sequences encoding a polypeptide that is at least 75% identical to the calcineurin modulators of the invention. The nucleic acid can be, e.g., a genomic DNA fragment, or it can be a cDNA molecule. The present invention is also directed to host cells transformed with a vector comprising an adapt78 nucleic acid molecule.

In one aspect, the invention includes a calcineurin modulator which is a purified Adapt78 protein or polypeptide, e.g., any of the Adapt78 polypeptides encoded by a Adapt78 nucleic acid, and fragments, homologs, analogs, and derivatives thereof. The invention also includes a pharmaceutical composition that includes a Adapt78 protein or polypeptide, and a pharmaceutically acceptable carrier or diluent.

The invention further provides a method for producing a calcineurin modulator. The method includes providing a cell containing a Adapt78 nucleic acid, e.g., a vector that includes a Adapt78 nucleic acid, and culturing the cell under conditions sufficient to express the peptide encoded by the nucleic acid. The expressed polypeptide is then recovered from the cell. The cell can be, e.g., a prokaryotic cell or eukaryotic cell. Preferably, a higher eukaryotic cell, e.g., mammalian is employed.

The calcineurin modulators of the invention include isolated peptides having the formula

X₁KQFLISPPASPPVX₂ (SEQ ID NO:1)

where X₁ and X₂, together, contain 0 to 200 amino acids, and the peptide has calcineurin modulating activity. Preferably, X₁ and X₂, together contain 0 to 100 amino acids, 0 to 50 amino acids, or 0 to 34 amino acids. One or more of the S residues may also be phosphorylated.

In another embodiment, the peptides of the invention may have the formula X₁Xaa₁Xaa₂FLISXaa₃Xaa₄AS Xaa₅Xaa₆ VX₂ (SEQ ID NO:7), where X₁ and X₂, together, contain 0 to 200 amino acids; Xaa₁ is lysine or arginine; Xaa₂ is glutamine, asparagine or glutamate; and Xaa₃, Xaa₄, Xaa₅, or Xaa₆ is proline or hydroxyproline. The peptide desirably has calcineurin modulating activity. Preferably, X₁ and X₂, together contain 0 to 100 amino acids, 0 to 50 amino acids, or 0 to 34 amino acids, and one or more of the S residues may be phosphorylated.

The calcineurin modulators of the invention, in another embodiment, include isolated peptides having the formula KQFLISPPASPPV (SEQ ID NO:2), or PDKQFLISPPASPPVGWKQVPKPKIIQTRRPE (SEQ ID NO: 8), where the peptide has calcineurin modulating activity. The peptides of the invention may also be joined or fused to other peptides, such as a HIV TAT 10-mer peptide, GRKKRRQRRR (SEQ ID NO:3). Such fusion proteins include an Adapt78 peptide inhibitor of calcineurin conjugated to the HIV-TAT protein for cell permeabilization, GRKKRRQRRRPPKQFLISPPASPPV (SEQ ID NO:4), or a fusion protein comprising SEQ ID NO: 2 and SEQ ID NO: 3.

Methods for determining candidate modulators of Adapt78 or Adapt78 peptides are also part of the invention. These methods include contacting a candidate modulator and an Adapt78 or Adapt78 peptide to a cell sample in which calcineurin is active, and determining the effect of the candidate modulator compared to the effect of the Adapt78 or Adapt78 peptide on the cell sample in the absence of the candidate modulator. Suitable cell samples include cells such as IMR-90 fibroblasts; U251 astroglioma cells; MCF7 breast adenocarcinoma cells; HeLa epitheliod carcinoma cells; HL60 promyelocytes; BEC(2)-M17 neuroblastoma cells; and primary mouse cardiomyocyte cells. Desirably the cells are human cells.

The invention also relates to transgenic animals, e.g., mice, that contain an adapt78 transgene that overexpress a human Adapt78 protein or a human Adapt78 peptide, e.g., the peptides described in this specification. These transgenic mice can be used to screen chemical compounds useful in the treatment of calcineurin-associated disorders, pathologies and abnormalities such as cardiac hypertrophy, memory loss, immune system dysfunction, and developmental disabilities; and can be used as a working model of Down Syndrome and related disorders such as developmental disorders. The invention further includes the method for making these transgenic mice.

Antibodies specific for Adapt78 or Adapt78 peptides are also within the scope of the invention, and are described in the Examples, including antibodies raised against the peptide of SEQ ID NO: 6, EMERMPKP.

The invention further includes polynucleotides encoding the polypeptides of the invention, vectors comprising these polynucleotides, including expression vectors, and host cells genetically engineered to express these polynucleotides. The host cells may be such that the polynucleotide is in operative association with a regulatory sequence that controls expression of the polynucleotide in the host cell.

The invention also includes pharmaceutical compositions or the like which comprise polypeptides of the invention, and a pharmaceutically acceptable carrier.

The invention further includes methods of modulating calcineurin activity in a subject, comprising administering one of the peptides of the invention to a subject, such that calcineurin activity is modulated, e.g., down regulated. Such methods further include treating Alzheimer's Disease, and calcineurin overexpression or overactivation conditions such as cancer, immune system and brain disorders, skeletal and cardiac muscle dysfunction, cardiac hypertrophy and heart failure. These methods are advantageous in one aspect because the peptides of the invention do not result in substantial adverse side effects to the patient, compared to conventional calcineurin modulators like FK506 or cyclosporin. The peptides of the invention may even be administered, in some cases, in conjunction with another calcineurin inhibitor.

Another aspect of the invention includes making and using a calcineurin modulator that can specifically target a particular part of the body.

### BRIEF DESCRIPTION OF THE DRAWING

FIG. 1 depicts the stable transfection of HA-1 cells with hamster adapt78 cDNA generating several sublines (designated 7, 10 and 15) overexpressing adapt78 mRNA.

FIG. 2 depicts the strong growth suppression observed for adapt78 overexpressors in HA-1 cells, compared with control.

FIG. 3 depicts significant suppression of the S-phase and G₂/M phase signals is observed in clone 15 cells with a concomitant increase in G₀/G₁-phase cells, as compared to control.

FIG. 4 depicts a study of adapt78 at the level of its protein product. An antibody was raised against an 8-mer peptide (EMERMPKP (SEQ ID NO: 6)) encoded for by the C-terminal region of Adapt78. In vitro transcription and translation, followed by Western blot analysis and a sensitive detection method (Pierce Super Signal Ultra) revealed that the Adapt78 antibody bound to in vitro transcription translation-generated Adapt78 protein (FIG. 4A), and that its binding to HA-1 cell lysate was inhibited by an excess of the Adapt78 eight amino acid epitope peptide (FIG. 4B). Thus, the obtained antibody (anti78) is specific to Adapt78. This antibody also binds to Adapt78 that is overexpressed in stable transfectants as is shown for clone 7 in FIG. 4C.

FIG. 5 depicts the cytoplasmic staining of HA-1 cells indicating the intracellular localization of Adapt78.

### DETAILED DESCRIPTION OF THE INVENTION

This invention relates to compositions that are useful for inhibiting and potentiating the activity of cellular calcineurin. The compositions include, but are not limited to, linear peptides (e.g., SEQ ID NOs: 1, 2, 4, 5, 6, 7 and 8), cyclic peptides, peptide analogs, peptidomimetics, combinatorial chemicals, and whole proteins. Such compositions can be used to treat calcineurin- and adapt78-related (including family members ZAKI-4/MCIP2, and DSCR1L2) pathologies such as cardiac, brain, immune system and developmental abnormalities; to protect human cells and tissues against stress damage based on the cytoprotective activity of Adapt78; and to modulate cell growth based on the growth-altering activity of Adapt78.

The features and other details of the invention will now be more particularly described with reference to the accompanying drawings and pointed out in the claims. It will be understood that particular embodiments described herein are shown by way of illustration and not as limitations of the invention. The principal features of this invention can be employed in various embodiments without departing from the scope of the invention. All parts and percentages are by weight unless otherwise specified.

Definitions

For convenience, certain terms used in the specification, examples, and appended claims are collected here.

“Adapt78” or “Adapt78 protein” includes Adapt78 protein; homologs thereof, including human—Adapt78/DSCR1/MCIP1 splice variants 1 and 4; ZAKI-4/MCIP2; and DSCR1L2; and invertebrate—CBP1 in C. neoformans and Rcn1p in S. cerevisiae; analogs thereof, including CBP1 in C. neoformans and Rcn1p in S. cerevisiae; and family members of Adapt78, and homologs and analogs of those family members.

“Calcineurin Modulator” or “Adapt78 polypeptide” includes an active portion or fragment of Adapt78 protein or a family member or homolog thereof; a modified full-length Adapt78 protein or family member or homolog thereof; linear peptides, cyclic peptides, peptide analogs, peptidomimetics, mimics, and combinatorial chemicals.

“Peptides of the invention” include Adapt78 and Calcineurin Modulators. “Peptide” as used herein, includes any structure comprised of two or more amino acids. For the most part, the peptides of this invention comprise fewer than 60 amino acids, and preferably fewer than 30 amino acids, and most preferably ranging from about 4 to 30 amino acids. Peptide, as used here and in the claims, is also intended to include analogs, derivatives, salts, retro-inverso isomers, mimics, mimetics, or peptidomimetics thereof. The peptides of the invention further include other peptide modifications, including analogs, derivatives and mimetics, that retain the ability of the modulator to alter cell proliferation as described herein. For example, a peptidic structure of a modulator of the invention may be further modified to increase its stability, bioavailability, solubility, etc. “Analog”, “derivative” and “mimetic” include molecules which mimic the chemical structure of a peptidic structure and retain the functional properties of the peptidic structure. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball, J. B. and Alewood, P. F. (1990) J. Mol. Recognition 3:55. Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) Peptidomimetic Design and Chemical Approaches to Peptide Metabolism in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

A “derivative” (e.g., a peptide or amino acid) includes forms in which one or more reaction groups on the compound have been derivatized with a substituent group. Examples of peptide derivatives include peptides in which an amino acid side chain, the peptide backbone, or the amino- or carboxy-terminus has been derivatized (e.g., peptidic compounds with methylated amide linkages). An “analog” of a compound X includes compounds which retain chemical structures necessary for functional activity, yet which also contains certain chemical structures which differ. An example of an analog of a naturally-occurring peptide is a peptide which includes one or more non-naturally-occurring amino acids.

A “mimetic” of a compound includes compounds in which chemical structures of the compound necessary for functional activity have been replaced with other chemical structures which mimic the conformation of the compound. Examples of peptidomimetics include peptidic compounds in which the peptide backbone is substituted with one or more benzodiazepine molecules (see e.g., James, G. L. et al. (1993) Science 260:1937-1942).

“Nucleic acid molecule” includes DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” include nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule includes one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, an isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

“Oligonucleotide” includes a series of linked nucleotide residues, the oligonucleotide having a sufficient number of nucleotide bases to be used in a PCR reaction. A short oligonucleotide sequence may be based on, or designed from, a genomic or cDNA sequence, and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at least 6 contiguous nucleotides, or a complement thereof. Oligonucleotides may be chemically synthesized and may be used as probes.

“Complementary” includes Watson-Crick or Hoogsteen base pairing between nucleotides units of a nucleic acid molecule. “Binding” includes the physical or chemical interaction between two polypeptides or compounds or associated polypeptides or compounds or combinations thereof. Binding includes ionic, non-ionic, Van der Waals, hydrophobic interactions, etc. A physical interaction can be either direct or indirect. Indirect interactions may be through or due to the effects of another polypeptide or compound. “Direct binding” includes interactions that do not take place through, or due to, the effect of another polypeptide or compound, but instead are without other substantial chemical intermediates.

“Fragments” include sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice.

“Derivatives” include nucleic acid sequences or amino acid sequences formed from the native compounds either directly or by modification or partial substitution.

“Analogs” include nucleic acid sequences or amino acid sequences that have a structure similar to, but not identical to, the native compound, but differs from the native compound in certain components or side chains. Analogs may be synthetic or from a different evolutionary origin, and may have a similar or opposite metabolic activity compared to wild type.

“Homologs” include nucleic acid sequences or amino acid sequences of a particular gene that are derived from different species.

Derivatives and analogs may be full length or other than full length, if the derivative or analog contains a modified nucleic acid or amino acid. Derivatives or analogs of the nucleic acids or proteins of the invention include molecules comprising regions that are substantially homologous to the nucleic acids or proteins of the invention, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

A “homologous nucleic acid sequence” or “homologous amino acid sequence,” or variations thereof, includes sequences characterized by a homology at the nucleotide level or amino acid level as discussed above. Homologous nucleotide sequences encode those sequences coding for isoforms of a polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a polypeptide of species other than humans, including, but not limited to, mammals, and thus can include, e.g., mouse, rat, rabbit, dog, cat cow, horse, and other organisms. Homologous nucleotide sequences also include naturally occurring allelic variations and mutations of the nucleotide sequences set forth herein. A homologous nucleotide sequence does not, however, include the nucleotide sequence encoding the protein. Homologous nucleic acid sequences include those nucleic acid sequences that encode conservative amino acid substitutions, as well as an active polypeptide.

An “open reading frame” (“ORF”) corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. An ORF may be any part of a coding sequence, with or without a start codon, a stop codon, or both. For an ORF to be considered as a good candidate for coding for a bona fide cellular protein, a minimum size requirement is often set, for example, a stretch of DNA that would encode a protein of 50 amino acids or more.

“Transgenic” includes cells that contain exogenous genetic material, or an animal that contains exogenous genetic material within most of the animal's cells. “Transgenic” also describes any transgenic technology known to those in the art that can produce a cell or animal carrying an introduced transgene; and a transgene created by providing an RNA that is transcribed into DNA, and then incorporated into the genome.

A “transgenic animal” includes non-animals, preferably mammals, more preferably rodents such as rats or mice, in which one or more of the cells of the animal includes a transgene. Other examples of transgenic animals include non-primates, sheep, dogs, cows, goats, chickens, amphibians, etc.

“Transgene” includes a piece of DNA that is inserted by artifice into a cell, and becomes part of the genome of the organism (either stably integrated or as a stable extrachromosomal element) that develops from that cell, thereby directing, e.g., the expression of an encoded gene product in one or more cell types or tissues of the transgenic animal. Transgenes of the invention include DNA polynucleotides that encode for the expression of human adapt78 mRNA and protein products; and polynucleotides that are important in the regulation of expression of the adapt78 gene.

A “homologous recombinant animal” includes non-animals, preferably mammals, more preferably mice, in which an endogenous gene, e.g., adapt78, has been altered by homologous recombination between the endogenous gene and an exogenous DNA molecule introduced into a cell of the animal, e.g., an embryonic cell of the animal, prior to development of the animal.

“Modulators” include compositions that alter (i.e., increase or decrease) the level of phosphatase activity in an in vitro assay, living cell and/or organism.

“Inhibitors” include compositions that lower or eliminate the level of phosphatase activity in an in vitro assay, living cell and/or organism, and are a type of modulator.

“Potentiators” include compositions that increase the level of phosphatase activity in an in vitro assay, living cell and/or organism, and are also a type of modulator. Thus, modulators include inhibitors and potentiators.

For convenience in describing polypeptides, the conventional abbreviations for the various common amino acids are used as generally accepted in the peptide art, as recommended by the IUPAC-IUB Commission on Biochemical Nomenclature, Biochem. J. 219:345 (1984). These abbreviations are incorporated herein by reference. The following is a list of some of these abbreviations:

| | Abbreviation |
|---|---|
| Alanine | A |
| Arginine | R |
| Asparagine | N |
| Aspartic acid | D |
| Cysteine | C |
| Glutamine | Q |
| Glutamic acid | E |
| Glycine | G |
| Histidine | H |
| Isoleucine | I |
| Leucine | L |
| Lysine | K |
| Methionine | M |
| Phenylalanine | F |
| Proline | P |
| Serine | S |
| Threonine | T |
| Tryptophan | W |
| Tyrosine | Y |
| Valine | V |

All peptide sequences mentioned herein are written according to the generally accepted convention whereby the N-terminal amino acid is on the left and the C-terminal amino acid is on the right. The abbreviations herein represent L-amino acids unless otherwise designated as D- or D,L-. Certain amino acids, both natural or non-natural, are achiral, e.g., glycine.

“Therapeutically effective amount” includes that amount of the biologically active polypeptide that will elicit the desired therapeutic and/or prophylactic effect (or utility).

Recent studies (e.g., Gorlach et al. mentioned above) demonstrate that Adapt78 protein, also referred to as DSCR1 and MCIP1, binds to and inhibits the action of cellular calcineurin. This binding was localized to a region (amino acids 338-352) between the catalytic domain and the regulatory B subunit-binding domain of the catalytic A subunit of calcineurin. Several human (Adapt78/DSCR1/MCIP1 splice variants 1 and 4, ZAKI-4/MCIP2, and DSCR1L2) and invertebrate (CBP1 in C. neoformans and Rcn1p in S. cerevisiae) homologs of Adapt78 exhibit this effect. Lower calcineurin binding by the Adapt78/DSCR1 yeast analog Rcn1 was associated with decreased calcineurin phosphatase activity inhibition, and overexpression of Adapt78/DSCR1 inhibited two calcineurin activities. While a specific 24 amino acid stretch of Adapt78 was identified as crucial to this inhibition, and even significantly inhibitory by itself, the full-length Adapt78 gave greater inhibition, possibly reflecting a conformational flexibility in the peptide that is not available in the whole protein. It appears that Adapt78 functions as a calcineurin feedback inhibitor, since calcineurin signaling strongly stimulates adapt78/DSCR1 transcription, and overexpression of Adapt78/DSCR1 inhibits calcineurin-dependent activation of the NF-AT transcription factor. The strong up-regulation of Adapt78/DSCR1 in response to calcineurin signaling differentiates Adapt78/DSCR1 from other calcineurin-interacting regulators including immunophilins, AKAP79, and cabin 1/cain.

adapt78 has also been identified as a stress-response gene using multiple stress agents including calcium ionophore A23187, hydrogen peroxide, 2-deoxyglucose, brefeldin A, tunicamycin, thapsigargin, and cyclopiazonic acid. adapt78 mRNA is 2.35 kb in size, induced as early as 90 minutes after stress agent exposure, and is strongly dependent upon calcium for its induction. The induction of adapt78 as a stress response gene was originally demonstrated in a so-called adaptive response model system using hamster cells in culture. In this system, pre-exposure of hamster cells to a mildly toxic concentration of hydrogen peroxide induced adapt78 and conferred protection against a higher concentration of peroxide, and concomitantly led to growth arrest. Thus, adapt78 induction correlated with stress protection. Consistent with this, subsequent analysis in these same cells indicated that adapt78 overexpression confers both stress protection and growth arrest as originally inferred by our adaptive response studies.

Many approaches have been used to design biologically active peptides, including combinatorial approaches, phage display approaches, and rational design approaches. For Adapt78 protein, a 24 amino acid stretch of Adapt78 protein was identified as crucial for calcineurin inhibition, as discussed above. For a peptide of this size, and about which a great deal of sequence homology information is available, the rational design approaches are very attractive. An example of this approach was in designing an anti-cancer peptide from alpha-fetoprotein (AFP) for treating breast cancer. See, e.g., Festin et al., Biochim Biophys Acta 1427:307-314 (1999) and Mesfin et al. Biochim Biophys Acta 1501:33-43 (2000). This work used a ‘parsing’ approach to decrease the active polypeptide size, first by expression of domains and subdomains, then by a synthetic peptide approach. The active polypeptide decreased in size from 68,000 MW for intact AFP, to 20,000 for Domain III, and then to 3860 for the first active synthetic peptide, a 34-mer. The 34-mer was then parsed further until an 8-mer peptide with full activity was identified (MW 842, or 1% of the intact protein). Subsequent design approaches were intended to increase shelf life and expand the effective dose range of the AFP-derived octapeptide, and included cyclization of the peptide. These strategies were successful in yielding a peptide analog with high potency, specificity, and with substantially more clinical translatability as a novel agent for breast cancer treatment.

Combinatorial chemistry is another approach that can be used to target intracellular proteins. In this approach, large numbers of compounds, sometimes millions, are generated and screened for their effect on the endpoint of interest, often binding to a macromolecular target or modulation of a biological response. The usual starting point for this approach is a chemical compound library. Screening this library for modulators of a target of interest generates so-called “hits”. The structures of compounds that hit can then be exploited by modification to produce combinatorial libraries. These second and third generation libraries are then screened to identify compounds of maximal potency. It is now possible to synthesize and screen very large numbers of compounds for drug discovery and development see, e.g., Leach et al. Drug Discov Today 5:326-336 (2000).

Given the wide range of cellular and physiological functions mediated by calcineurin, targeting Adapt78 represents a potential powerful therapeutic approach against any or all associated diseases and disorders. Calcineurin activity modulators described in this invention have potential therapeutic value for, e.g., the following:

Immune system. Calcineurin plays a central role in certain areas of immune system function including lymphocyte activation through stimulation of various lymphokines, cell surface receptor regulation, and apoptosis regulation. Thus, Adapt78-based inhibitors would have potential benefit in treating immune system dysfunction.

Heart disease. Cyclosporin and FK506, both of which act by inhibiting calcineurin, prevent cardiac hypertrophy in transgenic mice, but these agents are toxic in humans. Thus, Adapt78-based inhibitors would have potential benefit in preventing and treating cardiac hypertrophy and heart failure.

Brain. Calcineurin is highly abundant in the brain, and is known to be involved in many neuronal processes. These include long term potentiation and neurite outgrowth, which suggest a potential therapeutic role for Adapt78-based regulators would have potential benefit in promoting memory and recovery from neurological injury. For example, inhibition of calcineurin has been shown to promote neurite outgrowth; therefore, Adapt78-based inhibitors would have potential benefit in promoting neurological recovery.

Cancer. Calcineurin mediates certain types of apoptosis. Regulating apoptosis in cancer cells has become a useful anti-cancer approach. Thus, Adapt78-based inhibitors would have potential benefit as an anti-cancer treatment. adapt78 can also alter cell growth and thus may be useful in inhibiting cancer cell growth.

Transplantation. Cyclosporin and FK506 are potent immunosuppressants that inhibit graft rejection after organ transplantation. However, they both have undesirable side effects. An alternative form of immunosuppression, or a lowered concentration of cyclosporin/FK506 in combination with another agent, that would lower the toxic side effects would be beneficial. Like cyclosporin and FK506, almost all current immunosuppressive agents are calcineurin inhibitors. Thus, Adapt78-based inhibitors may be useful as an immunosuppressant.

Antifungal and antiparasitic agent. Calcineurin has been isolated from several human pathogens, and its presence has been shown to be required for virulence (e.g., the fungus C. neoformans). FK506 and cyclosporin, both calcineurin inhibitors, inhibit the growth of some of these pathogens, suggesting that Adapt78-based inhibition of calcineurin may have clinical application as an antifungal and antiparasitic agent as well as for understanding the role of calcineurin in fungal virulence.

Alzheimer's disease. A known target substrate of calcineurin is tau protein. An early event in Alzheimer's disease (AD) is the accumulation of stably phosphorylated tau, and this correlates with cognitive impairment. Calcineurin has been reported to regulate at least two phosphorylation sites on tau. A reduction in calcineurin phosphatase activity has also been reported in Alzheimer's, something that could be controlled by the Adapt78-based modulators of the invention—in this case, ones that attenuate calcineurin inhibition.

Cellular stress response. Overexpression of adapt78 prevents stress damage in cultured cells. Thus, the compositions described in this invention have potential utility in abrogating many different types of stress-related damage, such as ischemia-reperfusion injury damage and brain excitotoxicity. In addition, the success of certain clinical procedures might be improved by prior exposure of certain organs to Adapt78 mimics, an approach similar to the use of ischemic preconditioning prior to heart surgery. In addition, the adapt78 transgenic mice described herein represent an in vivo model system for studying the effects of human adapt78 in stress protection and growth arrest.

Down Syndrome. adapt78/DSCR1 is a chromosome 21 gene, and its mRNA has been reported to be overexpressed in human Down Syndrome brain. adapt78 is also located on chromosome 16 of Tn16 mice, a mouse model of adapt78 where critical genes involved in Down Syndrome reside. Down Syndrome is caused by trisomy 21, where an extra copy of genes on part of chromosome 21 is present in the cells of afflicted patients. Thus, adapt78 may be involved in the etiology of Down Syndrome and, if so, Adapt78-based regulators would be of potential clinical benefit. In addition, the adapt78 transgenic mice described herein represent a Down Syndrome model for use in studying and treating Down Syndrome and related developmental disorders.

Modulators of calcineurin activity have been discovered according to the present invention which are useful for treating calcineurin-related pathologies including, but not limited to, preventing and treating cardiac hypertrophy and heart failure; preventing and treating immune system dysfunction; preventing and treating brain dysfunction, including: promoting memory, recovery from neurological injury, and treating Alzheimer's disease; preventing and treating cancer via calcineurin's role in certain types of apoptosis; as immunosuppressive agents during transplantation; preventing and treating Down Syndrome; and as antifungal and antiparasitic agents. The composition of an Adapt78 peptide inhibitor of calcineurin is shown in SEQ ID NO:1:

X₁KQFLISPPASPPVX₂ (SEQ ID NO:1)

where X₁ contains between 0 and 200 amino acids and X₂ contains between 0 and 200 amino acids. These modulators may also include modifications of SEQ ID NO:1 by, e.g., cyclization, acetylation, and alkylation, or other chemical reaction. These modulators may further comprise amino acid substitution, including substituting D- for L-amino acids; substituting hydroxyproline for proline, substituting amino acids by those representing the oxidized residue (e.g., substituting a glutamic acid for a proline), and in any combination. These modulators may further comprise SEQ ID NO:1 or any of the described modified sequences, which is phosphorylated at one or both of the serine amino acid residues within the KQFLISPPASPPV (SEQ ID NO:2) domain, or at other phosphorylatable residues within the composition. The modulators may be generated using the synthetic methods described in the Examples.

Peptides of the Invention

The calcineurin modulators of the invention include isolated Peptides Of The Invention, and biologically active portions thereof, or derivatives, fragments, analogs or homologs thereof. Also provided are polypeptide fragments suitable for use as immunogens to raise anti-Adapt78 antibodies. In one embodiment, native Peptides Of The Invention can be isolated from cells or tissue sources by an appropriate purification scheme using standard protein purification techniques. In another embodiment, Peptides Of The Invention are produced by recombinant DNA techniques. A Peptide Of The Invention can be alternately be synthesized chemically using standard peptide synthesis techniques.

An “isolated” or “purified” protein or biologically active portion thereof is substantially free of cellular material or other contaminating proteins from the cell or tissue source from which the Peptide Of The Invention is derived, or substantially free from chemical precursors or other chemicals when chemically synthesized. “Substantially free of cellular material” includes preparations of a Peptide Of The Invention in which the protein is separated from cellular components of the cells from which it is isolated or recombinantly produced. In one embodiment, the language “substantially free of cellular material” includes preparations of a Peptide Of The Invention having less than about 30% (by dry weight) of non-Adapt78 protein (also referred to herein as a “contaminating protein”), more preferably less than about 20% of non-Adapt78 protein, still more preferably less than about 10% of non-Adapt78 protein, and most preferably less than about 5% non-Adapt78 protein. When the Adapt78 protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of a Peptide Of The Invention in which the protein is separated from chemical precursors or other chemicals that are involved in the synthesis of the protein. In one embodiment, the language “substantially free of chemical precursors or other chemicals” includes preparations of a Peptide Of The Invention having less than about 30% (by dry weight) of chemical precursors or non-Peptide chemicals, more preferably less than about 20% chemical precursors or non-Peptide chemicals, still more preferably less than about 10% chemical precursors or non-Peptide chemicals, and most preferably less than about 5% chemical precursors or non-Peptide chemicals.

Biologically active portions of a Peptide Of The Invention include peptides comprising amino acid sequences sufficiently homologous to or derived from the amino acid sequence of the Peptide Of The Invention that include fewer amino acids than the full length Peptides Of The Invention, and exhibit at least one activity of a Peptide Of The Invention. Typically, biologically active portions comprise a domain or motif with at least one activity of the Peptide Of The Invention. A biologically active portion of a Peptide Of The Invention can be a polypeptide which is, for example, 10, 25, 50, 100 or more amino acids in length.

A biologically active portion of a Peptide Of The Invention may contain at least one of the above-identified structural domains. Moreover, other biologically active portions, in which other regions of the protein are deleted, can be prepared by recombinant techniques and evaluated for one or more of the functional activities of a native Peptide Of The Invention.

In an embodiment, the Peptide Of The Invention has an amino acid sequence shown in SEQ ID NOs: 1, 2, 4, 5, 6, 7 or 8. In other embodiments, the Peptide Of The Invention is substantially homologous to SEQ ID NOs: 1, 2, 4, 5, 6, 7 or 8, and retains the functional activity of the protein of SEQ ID NOs: 1, 2, 4, 5, 6, 7 or 8, yet differs in amino acid sequence due to natural allelic variation or mutagenesis, as described in detail below. Accordingly, in another embodiment, the Peptide Of The Invention is a protein that comprises an amino acid sequence at least about 45% homologous to the amino acid sequence of SEQ ID NOs: 1, 2, 4, 5, 6, 7 or 8, and retains the functional activity of the Peptides Of The Invention of SEQ ID NOs: 1, 2, 4, 5, 6, 7 or 8.

Multimers

Also provided by the invention are protein multimers (i.e., polymers). A multimer includes, e.g., dimers, trimers, or tetramers. A multimer comprises a Peptide Of The Invention, or a biologically active portions thereof, or derivatives, fragments, analogs or homologs thereof, and a second polypeptide. The polypeptides of the multimer interact covalently, e.g, disulfide bond, or non-covalently. Alternatively, the polypeptides of the multimer may be chemically linked.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., amino acid or nucleic acid “homology” is equivalent herein to amino acid or nucleic acid “identity”).

The nucleic acid sequence homology may be determined as the degree of identity between two sequences. The homology may be determined using computer programs known in the art, such as GAP software provided in the GCG program package. See, e.g., Needleman and Wunsch (1970) J Mol Biol 48: 443-453.

“Sequence identity” includes the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides chimeric or fusion proteins. A “chimeric protein” or “fusion protein” comprises a Peptide Of The Invention operatively linked to a non-Peptide Of The Invention. Alternatively, the fusion protein is a multimer, e.g., homodimer or heterodimer. A “non-Peptide Of The Invention” refers to a polypeptide having an amino acid sequence corresponding to a protein that is not substantially homologous to the Peptide Of The Invention, e.g., a protein that is different from the Peptide Of The Invention and that is derived from the same or a different organism. Within a fusion protein the polypeptide can correspond to all or a portion of a Peptide Of The Invention. In one embodiment, a fusion protein comprises at least one biologically active portion of a Peptide Of The Invention. In another embodiment, a fusion protein comprises at least two biologically active portions of a Peptide Of The Invention. In yet another embodiment, a fusion protein comprises at least three biologically active portions of a Peptide Of The Invention. Within the fusion protein, the term “operatively linked” is intended to indicate that the Peptide Of The Invention and the non-Peptide Of The Invention are fused in-frame to each other. The non-Peptide Of The Invention can be fused to the N-terminus or C-terminus of the Peptide Of The Invention.

For example, in one embodiment a fusion protein comprises a Peptide Of The Invention operably linked to an HIV TAT peptide such as HIV TAT 10-mer peptide, GRKKRRQRRR (SEQ ID NO:3), as described in Example 5. In addition, the modulators of the invention may further be attached to a residue capable of targeting the modulator into cells and specific areas of the body (a targeting moiety), such as HIV TAT 10-mer peptide, GRKKRRQRRR (SEQ ID NO:3), as described in Example 5. One embodiment of the invention is an Adapt78 peptide inhibitor of calcineurin conjugated to the HIV-TAT protein for cell permeabilization is GRKKRRQRRRPPKQFLISPPASPPV (SEQ ID NO:4). The construction of an Adapt78-TAT fusion protein is described in Example 5.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Also, many expression vectors are commercially available that already encode a fusion moiety (e.g., a GST polypeptide). An Adapt78-encoding nucleic acid can be cloned into such an expression vector such that the fusion moiety is linked in-frame to the Adapt78 protein.

Agonists and Antagonists

The present invention also pertains to variants of the Peptides Of The Invention that function as either calcineurin agonists (mimetics) or as calcineurin antagonists. Variants of the Peptides Of The Invention can be generated by mutagenesis, e.g., discrete point mutation or truncation of the Peptides Of The Invention. An agonist of the protein can retain substantially the same, or a subset of, the biological activities of the naturally occurring form of the protein. An antagonist of calcineurin can inhibit one or more of the activities of the naturally occurring form of the Peptides Of The Invention by, for example, competitively binding to a downstream or upstream member of a cellular signaling cascade which includes the Peptides Of The Invention. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the Peptides Of The Invention.

Variants of the Peptides Of The Invention that function as either calcineurin agonists (mimetics) or as calcineurin antagonists can be identified by screening combinatorial libraries of mutants, e.g., truncation mutants, of the Peptides Of The Invention for agonist or antagonist activity. In one embodiment, a variegated library of Peptide variants is generated by combinatorial mutagenesis at the nucleic acid level and is encoded by a variegated gene library. A variegated library of variants of the Peptides Of The Invention can be produced by, for example, enzymatically ligating a mixture of synthetic oligonucleotides into gene sequences such that a degenerate set of potential sequences is expressible as individual polypeptides, or alternatively, as a set of larger fusion proteins (e.g., for phage display) containing the set of sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the sequence coding for Peptides Of The Invention can be used to generate a variegated population of fragments for screening and subsequent selection of variants of a Peptide Of The Invention. In one embodiment, a library of coding sequence fragments can be generated by treating a double stranded PCR fragment of a coding sequence with a nuclease under conditions wherein nicking occurs only about once per molecule, denaturing the double stranded DNA, re-annealing the DNA to form double stranded DNA that can include sense/antisense pairs from different nicked products, removing single stranded portions from reformed duplexes by treatment with S1 nuclease, and ligating the resulting fragment library into an expression vector. An expression library can therefore be derived which encodes N-terminal and internal fragments of various sizes of the Peptide Of The Invention.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of Peptides Of The Invention. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify variants (Arkin and Yourvan (1992) PNAS 89:7811-7815; Delgrave et al. (1993) Protein Engineering 6:327-331).

Anti-Adapt78 Antibodies

The invention encompasses antibodies and antibody fragments that bind immunospecifically to any of the polypeptides. An isolated Peptide Of The Invention, or a portion or fragment thereof, can be used as an immunogen to generate antibodies that bind Adapt78 using standard techniques for polyclonal and monoclonal antibody preparation. The full-length protein can be used or, alternatively, the invention provides antigenic peptide fragments of Adapt78 for use as immunogens, e.g., an 8 amino acid stretch near the C-terminus (EMERMPKP (SEQ ID NO: 6)). The antigenic peptide of Adapt78 encompasses an epitope of Adapt78 such that an antibody raised against the peptide forms a specific immune complex with Adapt78. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

As disclosed herein, Adapt78 protein sequences, or derivatives, fragments, analogs or homologs thereof, may be utilized as immunogens in the generation of antibodies that immunospecifically-bind these protein components. “Antibody” includes immunoglobulin molecules and immunologically active portions of immunoglobulin molecules, i.e., molecules that contain an antigen binding site that specifically binds (immunoreacts with) an antigen, such as Adapt78. Such antibodies include, but are not limited to, polyclonal, monoclonal, chimeric, single chain, F_{ab} and F_{(ab′)2} fragments, and an F_{ab} expression library. In a specific embodiment, antibodies to Peptides Of The Invention are disclosed. Various procedures known within the art may be used for the production of polyclonal or monoclonal antibodies to a Adapt78 protein sequence, or derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed Adapt78 protein or a chemically synthesized Adapt78 polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants such as Bacille Calmette-Guerin and Corynebacterium parvum, or similar immunostimulatory agents. If desired, the antibody molecules directed against Adapt78 can be isolated from the mammal (e.g., from the blood) and further purified by well known techniques, such as protein A chromatography to obtain the IgG fraction.

“Monoclonal antibody” or “monoclonal antibody composition”, includes populations of antibody molecules that contain only one species of an antigen binding site capable of immunoreacting with a particular epitope of Adapt78. A monoclonal antibody composition thus typically displays a single binding affinity for a particular Adapt78 protein with which it immunoreacts. For preparation of monoclonal antibodies directed towards a particular Adapt78 protein, or derivatives, fragments, analogs or homologs thereof, any technique that provides for the production of antibody molecules by continuous cell line culture may be utilized. Such techniques include the hybridoma technique (see Kohler & Milstein, 1975 Nature 256: 495-497); the trioma technique; the B-cell hybridoma technique (see Kozbor, et al., 1983 Immunol Today 4: 72) and the EBV hybridoma technique to produce monoclonal antibodies (see Cole, et al., 1985 In: Monoclonal Anitbodies and Cancer Therapy, Alan R. Liss, Inc., pp. 77-96). Monoclonal antibodies may be utilized in the practice of the invention and may be produced using hybridomas (see Cote, et al., 1983. Proc Natl Acad Sci USA 80: 2026-2030) or by transforming B-cells with Epstein Barr Virus in vitro (see Cole, et al., 1985 In: Monoclonal Antibodies and Cancer Therapy, Alan R. Liss, Inc., pp. 77-96).

According to the invention, techniques can be adapted for the production of single-chain antibodies specific to a Adapt78 protein (see e.g., U.S. Pat. No. 4,946,778). In addition, methodologies can be adapted for the construction of F_{ab} expression libraries (see e.g., Huse, et al., 1989 Science 246: 1275-1281) to allow rapid and effective identification of monoclonal F_{ab} fragments with the desired specificity for a Adapt78 protein or derivatives, fragments, analogs or homologs thereof. Non-antibodies can be “humanized” by techniques well known in the art. See e.g., U.S. Pat. No. 5,225,539. Antibody fragments that contain the idiotypes to a Adapt78 protein may be produced by techniques known in the art including: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-Adapt78 antibodies, such as chimeric and humanized monoclonal antibodies, comprising both and non-portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985) Nature 314:446-449; Shaw et al. (1988) J Natl Cancer Inst 80:1553-1559); Morrison(1985) Science 229:1202-1207; Oi et al. (1986) BioTechniques 4:214; Jones et al. (1986) Nature 321:552-525; Verhoeyan et al. (1988) Science 239:1534; and Beidler et al. (1988) J Immunol 141:4053-4060.

In one embodiment, methodologies for screening antibodies that possess the desired specificity include enzyme-linked immunosorbent assay (ELISA) and other immunologically-mediated techniques known in the art. In a specific embodiment, selection of antibodies that are specific to a particular domain of a Adapt78 protein is facilitated by generation of hybridomas that bind to the fragment of a Adapt78 protein possessing such a domain.

Anti-Adapt78 antibodies may be used in methods known within the art relating to the localization and/or quantitation of an Adapt78 protein (e.g., for use in measuring levels of the Adapt78 protein within appropriate physiological samples, for use in diagnostic methods, for use in imaging the protein, and the like). In a given embodiment, antibodies for Peptides Of The Invention, or derivatives, fragments, analogs or homologs thereof, that contain the antibody derived binding domain, are utilized as pharmacologically-active compounds, or “therapeutics”.

An anti-Adapt78 antibody (e.g., monoclonal antibody) can be used to isolate Adapt78 by standard techniques, such as affinity chromatography or immunoprecipitation. An anti-Adapt78 antibody can facilitate the purification of natural Adapt78 from cells and of recombinantly produced Adapt78 expressed in host cells. Moreover, an anti-Adapt78 antibody can be used to detect Adapt78 protein (e.g., in a cellular lysate or cell supernatant) in order to evaluate the abundance and pattern of expression of the Adapt78 protein. Anti-Adapt78 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, □-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Nucleic Acids

One aspect of the invention pertains to isolated nucleic acid molecules that encode Peptides Of The Invention or biologically active portions thereof, as well as nucleic acid fragments sufficient for use as hybridization probes to identify Adapt78-encoding nucleic acids (e.g., Adapt78 mRNA) and fragments for use as PCR primers for the amplification or mutation of Adapt78 nucleic acid molecules.

A nucleic acid molecule of the invention or a complement of any of these nucleotide sequences, can be isolated using standard molecular biology techniques and the sequence information provided herein. Using all or a portion of the nucleic acid sequence as a hybridization probe, Adapt78 molecules can be isolated using standard hybridization and cloning techniques (e.g., as described in Sambrook et al., (eds.), Molecular Cloning: A Laboratory Manual 2^{nd} Ed., Cold Spring Harbor Laboratory Press, Cold Spring Harbor, N.Y., 1989; and Ausubel, et al., (eds.), Current Protocols in Molecular Biology, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The amplified nucleic acid can be cloned into an appropriate vector and characterized by DNA sequence analysis. Furthermore, oligonucleotides corresponding to Adapt78 nucleotide sequences can be prepared by standard synthetic techniques, e.g., using an automated DNA synthesizer.

In another embodiment, an isolated nucleic acid molecule of the invention comprises a nucleic acid molecule that is a complement of the nucleotide sequence that encodes Peptides Of The Invention. In another embodiment, an isolated nucleic acid molecule of the invention comprises a nucleic acid molecule that is a complement of the nucleotide sequence that encodes Peptides Of The Invention, or a portion of this nucleotide sequence. A nucleic acid molecule that is complementary is one that is sufficiently complementary to the target nucleotide sequence that it can hydrogen bond with few or no mismatches, thereby forming a stable duplex.

Alternatively, an isolated nucleic acid molecule of the invention, e.g., an adapt78 nucleic acid, comprises contiguous nucleotides encoding the amino acid sequence of SEQ ID NOs: 1, 2, 4, 5, 6, 7 or 8.

An Adapt78 polypeptide is encoded by the open reading frame (“ORF”) of an Adapt78 nucleic acid. The nucleotide sequence of the adapt78 gene allows for the generation of probes and primers designed for use in identifying and/or cloning adapt78 homologs in other cell types, e.g. from other tissues, as well as adapt78 homologs from other mammals. The probe/primer typically comprises substantially purified oligonucleotide.

Probes based on the adapt78 nucleotide sequence can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissue which misexpress an Adapt78 protein, such as by measuring a level of an Adapt78-encoding nucleic acid in a sample of cells from a subject e.g., detecting adapt78 mRNA levels or determining whether a genomic adapt78 gene has been mutated or deleted.

“A polypeptide having a biologically active portion of Adapt78” refers to polypeptides exhibiting activity similar, but not necessarily identical to, an activity of a polypeptide of the invention, including mature forms, as measured in a particular biological assay, with or without dose dependency. A nucleic acid fragment encoding a “biologically active portion of Adapt78” can be prepared by isolating a portion of a nucleotide that encodes a polypeptide having an Adapt78 biological activity (the biological activities of the Peptides Of The Invention are described below), expressing the encoded portion of Adapt78 protein (e.g., by recombinant expression in vitro) and assessing the activity of the encoded portion of Adapt78.

Variants

The invention further encompasses nucleic acid molecules that differ from a given nucleotide sequence due to degeneracy of the genetic code and thus encode the same Peptide Of The Invention as that encoded by that given nucleotide sequence.

It will be appreciated by those skilled in the art that DNA sequence polymorphisms that lead to changes in the amino acid sequences may exist within a population (e.g., the population). Such genetic polymorphism in the adapt78 gene may exist among individuals within a population due to natural allelic variation. “Gene” and “recombinant gene” include nucleic acid molecules comprising an open reading frame encoding a protein, preferably a mammalian protein. Such natural allelic variations can typically result in 1-5% variance in the nucleotide sequence of the adapt78 gene. Any and all such nucleotide variations and resulting amino acid polymorphisms in adapt78 that are the result of natural allelic variation and that do not alter the functional activity of adapt78 are intended to be within the scope of the invention.

Moreover, nucleic acid molecules encoding Peptides Of The Invention from other species, and thus that have a nucleotide sequence that differs from a given sequence are intended to be within the scope of the invention. Nucleic acid molecules corresponding to natural allelic variants and homologs of the adapt78 cDNAs of the invention can be isolated based on their homology to the adapt78 nucleic acids disclosed herein using the cDNAs, or a portion thereof, as a hybridization probe according to standard hybridization techniques under stringent hybridization conditions.

“Hybridizes under stringent conditions” includes conditions for hybridization and washing under which nucleotide sequences at least 60% homologous to each other typically remain hybridized to each other.

Homologs (i.e., nucleic acids encoding Peptides Of The Invention derived from species other than human) or other related sequences (e.g., paralogs) can be obtained by low, moderate or high stringency hybridization with all or a portion of the particular sequence as a probe using methods well known in the art for nucleic acid hybridization and cloning.

“Stringent hybridization conditions” includes conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration) at which 50% of the probes complementary to the target sequence hybridize to the target sequence at equilibrium. Since the target sequences are generally present at excess, at Tₘ, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequence of a nucleotide that encodes for a Peptide Of The Invention and corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, Current Protocols in Molecular Biology, John Wiley & Sons, NY, and Kriegler, 1990, Gene Transfer and Expression, A Laboratory Manual, Stockton Press, NY.

A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, Current Protocols in Molecular Biology, John Wiley & Sons, NY, and Kriegler, 1990, Gene Transfer and Expression, A Laboratory Manual, Stockton Press, NY; Shilo and Weinberg, 1981, Proc Natl Acad Sci USA 78: 6789-6792.

Conservative Mutations

In addition to naturally-occurring allelic variants that may exist in the population, the skilled artisan will further appreciate that changes can be introduced by mutation into a nucleotide sequence, thereby leading to changes in the amino acid sequence of the encoded Adapt78 protein, without altering the functional ability of the Adapt78 protein. For example, nucleotide substitutions leading to amino acid substitutions at “non-essential” amino acid residues can be made in the sequence. A “non-essential” amino acid residue includes a residue that can be altered from the wild-type sequence without altering the biological activity, whereas an “essential” amino acid residue is required for biological activity.

Another aspect of the invention pertains to nucleic acid molecules encoding Peptides Of The Invention that contain changes in amino acid residues that are not essential for activity. Such Peptides Of The Invention differ in amino acid sequence from SEQ ID NOs: 1, 2, 5, 6 or 7, yet retain biological activity. In one embodiment, the isolated nucleic acid molecule comprises a nucleotide sequence encoding a protein, wherein the protein comprises an amino acid sequence at least about 45% homologous to the amino acid sequence of SEQ ID NOs: 1, 2, 5, 6 or 7. Preferably, the protein encoded by the nucleic acid molecule is at least about 60% homologous, more preferably at least about 70% homologous, still more preferably at least about 80% homologous, even more preferably at least about 90% homologous, and most preferably at least about 95% homologous.

Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” includes those in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in, e.g., Adapt78 is replaced with another amino acid residue from the same side chain family. Alternatively, in another embodiment, mutations can be introduced randomly along all or part of an, e.g., Adapt78 coding sequence, such as by saturation mutagenesis, and the resultant mutants can be screened for, e.g., Adapt78 biological activity to identify mutants that retain activity. Following mutagenesis of the polynucleotide, the encoded protein can be expressed by any recombinant technology known in the art and the activity of the protein can be determined.

Antisense

Another aspect of the invention pertains to isolated antisense nucleic acid molecules that are hybridizable to or complementary to the nucleic acid molecule comprising the nucleotide sequence encoding the Peptides Of The Invention, or fragments, analogs or derivatives thereof. An “antisense” nucleic acid includes a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire, e.g., Adapt78 coding strand, or to only a portion thereof. Nucleic acid molecules encoding fragments, homologs, derivatives and analogs of an, e.g., Adapt78 protein of SEQ ID NOs: 1, 2, 5, 6 or 7, or antisense nucleic acids complementary to an, e.g., Adapt78 nucleic acid sequence, are part of the invention.

In one embodiment, an antisense nucleic acid molecule is antisense to a “coding region” of the coding strand of a nucleotide sequence encoding, e.g., Adapt78. “Coding region” includes the region of the nucleotide sequence comprising codons which are translated into amino acid residues. In another embodiment, the antisense nucleic acid molecule is antisense to a “noncoding region” of the coding strand of a nucleotide sequence encoding, e.g., Adapt78. “Noncoding region” includes 5′ and 3′ sequences which flank the coding region that are not translated into amino acids (i.e., also referred to as 5′ and 3′ untranslated regions).

Antisense nucleic acids of the invention can be designed according to the rules of Watson and Crick or Hoogsteen base pairing. The antisense nucleic acid molecule can be complementary to the entire coding region of, e.g., Adapt78 mRNA, but more preferably is an oligonucleotide that is antisense to only a portion of the coding or noncoding region of, e.g., Adapt78 mRNA. For example, the antisense oligonucleotide can be complementary to the region surrounding the translation start site of, e.g., Adapt78 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an, e.g., Adapt78 protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of an antisense nucleic acid molecule administration route includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an □-anomeric nucleic acid molecule. An □-anomeric nucleic acid molecule forms specific double-stranded hybrids with complementary RNA in which, contrary to the usual □-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Ribozymes and PNA Moieties

Nucleic acid modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave, e.g., Adapt78 mRNA transcripts to thereby inhibit translation of, e.g., Adapt78 mRNA. A ribozyme having specificity for an, e.g., Adapt78 -encoding nucleic acid can be designed based upon the nucleotide sequence of an, e.g., Adapt78 cDNA. For example, a derivative of a Tetrahymena L-19 IVS RNA can be constructed in which the nucleotide sequence of the active site is complementary to the nucleotide sequence to be cleaved in an, e.g., Adapt78 -encoding mRNA. See, e.g., Cech et al. U.S. Pat. No. 4,987,071; and Cech et al. U.S. Pat. No. 5,116,742. Alternatively, e.g., Adapt78 mRNA can be used to select a catalytic RNA having a specific ribonuclease activity from a pool of RNA molecules. See, e.g., Bartel et al., (1993) Science 261:1411-1418.

Alternatively, e.g., Adapt78 gene expression can be inhibited by targeting nucleotide sequences complementary to the regulatory region of, e.g., Adapt78 (e.g., the Adapt78 promoter and/or enhancers) to form triple helical structures that prevent transcription of the, e.g., Adapt78 gene in target cells. See generally, Helene (1991) Anticancer Drug Des. 6: 569-84; Helene et al. (1992) Ann. N.Y Acad. Sci. 660:27-36; and Maher (1992) Bioassays 14: 807-15.

In various embodiments, the nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). “Peptide nucleic acids” or “PNAs” include nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of, e.g., Adapt78 can be used in therapeutic and diagnostic applications. For example, PNAs can be used as antisense or antigene agents for sequence-specific modulation of gene expression by, e.g., inducing transcription or translation arrest or inhibiting replication. PNAs of, e.g., Adapt78 can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of, e.g., Adapt78 can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of, e.g., Adapt78 can be generated that may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA SEQment and a 3′ DNA SEQment (Finn et al. (1996) above). Alternatively, chimeric molecules can be synthesized with a 5′ DNA SEQment and a 3′ PNA SEQment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. W088/09810) or the blood-brain barrier (see, e.g., PCT Publication No. W089/10134). Oligonucleotides can also be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

Nucleotide Polymorphisms Associated with, e.g., Adapt78 Genes

The invention also includes nucleic acid sequences that include one or more polymorphic, e.g., Adapt78 sequences. Also included are methods of identifying a base occupying a polymorphic in an, e.g., Adapt78 sequence, as well as methods of identifying an individualized therapeutic agent for treating, e.g., Adapt78 associated pathologies based on, e.g., Adapt78 sequence polymorphisms.

The nucleotide polymorphism can be a single nucleotide polymorphism (SNP). A SNP occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

In some embodiments, the polymorphic sequence includes the full length sequence. In other embodiments, the polymorphic sequence includes a polynucleotide that is between 10 and 100 nucleotides, 10 and 75 nucleotides, 10 and 50 nucleotides, or 10 and 25 nucleotides in length.

Adapt78 Recombinant Expression Vectors and Host Cells

Another aspect of the invention pertains to vectors, preferably expression vectors, containing a nucleic acid encoding, e.g., Adapt78 protein, e.g., Adapt78 multimers, or derivatives, fragments, analogs or homologs thereof. “Vector” includes nucleic acid molecules capable of transporting another nucleic acid to which it has been linked. One type of vector is a “plasmid”, a circular double stranded DNA loop into which additional DNA SEQments can be ligated. Another type of vector is a viral vector, wherein additional DNA SEQments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. “Plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). “Regulatory sequence” includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., Peptides Of The Invention, mutants, fusion proteins, etc.).

The recombinant expression vectors of the invention can be designed for expression of, e.g., Adapt78 in prokaryotic or eukaryotic cells. For example, e.g., Adapt78 can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11 d (Studier et al., Gene Expression Technology: Methods in Enzymology 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the, e.g., Adapt78 expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, e.g., Adapt78 can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention may be expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., Molecular Cloning: A Laboratory Manual 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the □-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to, e.g., Adapt78 mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, e.g., Adapt78 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. “Transformation” and “transfection” include a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding, e.g., Adapt78 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express), e.g., Adapt78 protein. Accordingly, the invention further provides methods for producing, e.g., Adapt78 protein using the host cells of the invention. In one embodiment, the method comprises culturing the host cell of invention (into which a recombinant expression vector encoding, e.g., Adapt78 has been introduced) in a suitable medium such that, e.g., Adapt78 protein is produced. In another embodiment, the method further comprises isolating, e.g., Adapt78 from the medium or the host cell.

Uses and Methods of the Invention

The isolated nucleic acid molecules of the invention can be used to express, e.g., Adapt78 protein (e.g., via a recombinant expression vector in a host cell in gene therapy applications), to detect, e.g., Adapt78 mRNA (e.g., in a biological sample) or a genetic lesion in a, e.g., Adapt78 gene, and to modulate, e.g., Adapt78 activity, as described further below. In addition, the Peptides Of The Invention can be used to screen drugs or compounds that modulate the, e.g., Adapt78 polypeptide, multimer or nucleic acid activity or expression as well as to treat disorders characterized by insufficient or excessive production of, e.g., Adapt78 protein or multimers or production of, e.g., Adapt78 protein or multimer forms that have decreased or aberrant activity compared to, e.g., Adapt78 wild type protein or multimer (e.g. proliferative disorders such as cancer, ovulatory disorders, infertility, hypogonadism or metabolic disorder effecting pituitary function or pituitary target organs such as for example, adrenal gland, thyroid, gonad or liver). In addition, the anti-Adapt78 antibodies of the invention can be used to detect and isolate Peptides Of The Invention and modulate, e.g., Adapt78 activity.

This invention further pertains to novel agents identified by the above described screening assays and uses thereof for treatments as described herein.

Screening Assays

The invention provides methods (also referred to “screening assays”) for identifying modulators, i.e., candidate or test compounds or agents (e.g., peptides, peptidomimetics, small molecules or other drugs) that bind to Peptides Of The Invention or, e.g., Adapt78 multimers or have a stimulatory or inhibitory effect on, for example, e.g., Adapt78 expression or activity.

In one embodiment, the invention provides assays for screening candidate or test compounds which bind to or modulate the activity of the membrane-bound form of a, e.g., Adapt78 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of, e.g., Adapt78 protein or, e.g., Adapt78 multimer, or a biologically active portion thereof, on the cell surface is contacted with a test compound and the ability of the test compound to bind to a, e.g., Adapt78 protein or multimer is determined. The cell, for example, can of mammalian origin or a yeast cell. Determining the ability of the test compound to bind to the, e.g., Adapt78 protein or multimer can be accomplished, for example, by coupling the test compound with a radioisotope or enzymatic label such that binding of the test compound to the, e.g., Adapt78 protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of, e.g., Adapt78 protein, or a biologically active portion thereof, on the cell surface with a known compound which binds, e.g., Adapt78 to form an assay mixture, contacting the assay mixture with a test compound, and determining the ability of the test compound to interact with a, e.g., Adapt78 protein, wherein determining the ability of the test compound to interact with a, e.g., Adapt78 protein comprises determining the ability of the test compound to preferentially bind to, e.g., Adapt78 or a biologically active portion thereof as compared to the known compound.

In another embodiment, an assay is a cell-based assay comprising contacting a cell expressing a membrane-bound form of, e.g., Adapt78 protein, or multimer or a biologically active portion thereof, on the cell surface with a test compound and determining the ability of the test compound to modulate (e.g., stimulate or inhibit) the activity of the, e.g., Adapt78 protein or multimer or biologically active portion thereof. Determining the ability of the test compound to modulate the activity of, e.g., Adapt78 or a biologically active portion thereof can be accomplished, for example, by determining the ability of the, e.g., Adapt78 protein to bind to or interact with a, e.g., Adapt78 target molecule. A “target molecule” includes molecules with which an, e.g., Adapt78 protein binds or interacts in nature, for example, calcineurin, or a molecule on the surface of a cell which expresses an, e.g., Adapt78 interacting protein, a molecule on the surface of a second cell, a molecule in the extracellular milieu, a molecule associated with the internal surface of a cell membrane or a cytoplasmic molecule. An, e.g., Adapt78 target molecule can be a non-Adapt78 molecule or a, e.g., Adapt78 protein or polypeptide of the invention. In one embodiment, an, e.g., Adapt78 target molecule is a component of a signal transduction pathway that facilitates transduction of an extracellular signal (e.g. a signal generated by binding of a compound to a membrane-bound, e.g., Adapt78 molecule) through the cell membrane and into the cell. The target, for example, can be a second intercellular protein that has catalytic activity or a protein that facilitates the association of downstream signaling molecules with, e.g., Adapt78.

Determining the ability of the, e.g., Adapt78 protein to bind to or interact with a, e.g., Adapt78 target molecule can be accomplished by one of the methods described above for determining direct binding. In one embodiment, determining the ability of the, e.g., Adapt78 protein to bind to or interact with a, e.g., Adapt78 target molecule can be accomplished by determining the activity of the target molecule. For example, the activity of the target molecule can be determined by detecting induction of a cellular second messenger of the target (i.e. intracellular Ca²+, diacylglycerol, IP₃, etc.), detecting catalytic/enzymatic activity of the target an appropriate substrate, detecting the induction of a reporter gene (comprising an, e.g., Adapt78-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a, e.g., Adapt78 protein or biologically active portion thereof with a test compound and determining the ability of the test compound to bind to the, e.g., Adapt78 protein or biologically active portion thereof. Binding of the test compound to the, e.g., Adapt78 protein can be determined either directly or indirectly as described above. In one embodiment, the assay comprises contacting the, e.g., Adapt78 protein or biologically active portion thereof with a known compound which binds, e.g., Adapt78 to form an assay mixture, contacting the assay mixture with a test compound, and determining the ability of the test compound to interact with an, e.g., Adapt78 protein, wherein determining the ability of the test compound to interact with an, e.g., Adapt78 protein comprises determining the ability of the test compound to preferentially bind to, e.g., Adapt78 or biologically active portion thereof as compared to the known compound.

In another embodiment, an assay is a cell-free assay comprising contacting, e.g., Adapt78 protein or biologically active portion thereof with a test compound and determining the ability of the test compound to modulate (e.g. stimulate or inhibit) the activity of the, e.g., Adapt78 protein or biologically active portion thereof. Determining the ability of the test compound to modulate the activity of, e.g., Adapt78 can be accomplished, for example, by determining the ability of the, e.g., Adapt78 protein to bind to a, e.g., Adapt78 target molecule by one of the methods described above for determining direct binding. In an alternative embodiment, determining the ability of the test compound to modulate the activity of, e.g., Adapt78 can be accomplished by determining the ability of the, e.g., Adapt78 protein further modulate an, e.g., Adapt78 target molecule. For example, the catalytic/enzymatic activity of the target molecule on an appropriate substrate can be determined as previously described.

In yet another embodiment, the cell-free assay comprises contacting the, e.g., Adapt78 protein or biologically active portion thereof with a known compound which binds, e.g., Adapt78 to form an assay mixture, contacting the assay mixture with a test compound, and determining the ability of the test compound to interact with a, e.g., Adapt78 protein, wherein determining the ability of the test compound to interact with a, e.g., Adapt78 protein comprises determining the ability of the, e.g., Adapt78 protein to preferentially bind to or modulate the activity of a, e.g., Adapt78 target molecule.

The cell-free assays of the present invention are amenable to use of both the soluble form or the membrane-bound form of, e.g., Adapt78. In the case of cell-free assays comprising the membrane-bound form of, e.g., Adapt78, it may be desirable to utilize a solubilizing agent such that the membrane-bound form of, e.g., Adapt78 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either, e.g., Adapt78 or its target molecule to facilitate separation of complexed from uncomplexed forms of one or both of the proteins, as well as to accommodate automation of the assay. Binding of a test compound to, e.g., Adapt78, or interaction of, e.g., Adapt78 with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-Adapt78 fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or, e.g., Adapt78 protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of, e.g., Adapt78 binding or activity determined using standard techniques.

Other techniques for immobilizing proteins on matrices can also be used in the screening assays of the invention. For example, either, e.g., Adapt78 or its target molecule can be immobilized utilizing conjugation of biotin and streptavidin. Biotinylated, e.g., Adapt78 or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with, e.g., Adapt78 or target molecules, but which do not interfere with binding of the, e.g., Adapt78 protein to its target molecule, can be derivatized to the wells of the plate, and unbound target or, e.g., Adapt78 trapped in the wells by antibody conjugation. Methods for detecting such complexes, in addition to those described above for the GST-immobilized complexes, include immunodetection of complexes using antibodies reactive with the, e.g., Adapt78 or target molecule, as well as enzyme-linked assays that rely on detecting an enzymatic activity associated with the, e.g., Adapt78 or target molecule.

In another embodiment, modulators of, e.g., Adapt78 expression are identified in a method wherein a cell is contacted with a candidate compound and the expression of, e.g., Adapt78 mRNA or protein in the cell is determined. The level of expression of, e.g., Adapt78 mRNA or protein in the presence of the candidate compound is compared to the level of expression of, e.g., Adapt78 mRNA or protein in the absence of the candidate compound. The candidate compound can then be identified as a modulator of, e.g., Adapt78 expression based on this comparison. For example, when expression of, e.g., Adapt78 mRNA or protein is greater (statistically significantly greater) in the presence of the candidate compound than in its absence, the candidate compound is identified as a stimulator of, e.g., Adapt78 mRNA or protein expression. Alternatively, when expression of, e.g., Adapt78 mRNA or protein is less (statistically significantly less) in the presence of the candidate compound than in its absence, the candidate compound is identified as an inhibitor of, e.g., Adapt78 mRNA or protein expression. The level of, e.g., Adapt78 mRNA or protein expression in the cells can be determined by methods described herein for detecting, e.g., Adapt78 mRNA or protein.

In yet another aspect of the invention, the Peptides Of The Invention can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with, e.g., Adapt78 (“Adapt78-binding proteins” or “Adapt78-bp”) and modulate, e.g., Adapt78 activity. Such, e.g., Adapt78-binding proteins are also likely to be involved in the propagation of signals by the Peptides Of The Invention as, for example, upstream or downstream elements of the, e.g., Adapt78 pathway.

The two-hybrid system is based on the modular nature of most transcription factors, which consist of separable DNA-binding and activation domains. Briefly, the assay utilizes two different DNA constructs. In one construct, the gene that codes for, e.g., Adapt78 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an, e.g., Adapt78-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with, e.g., Adapt78.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining, e.g., Adapt78 protein, e.g., Adapt78 multimer and/or nucleic acid expression as well as, e.g., Adapt78 or, e.g., Adapt78 multimer activity, in the context of a biological sample (e.g., blood, serum, cells, tissue) to thereby determine whether an individual is afflicted with a disease or disorder, or is at risk of developing a disorder, associated with aberrant, e.g., Adapt78 expression or activity, e.g. Alzheimer's Disease. The invention also provides for prognostic (or predictive) assays for determining whether an individual is at risk of developing a disorder associated with, e.g., Adapt78 protein expression or activity. For example, mutations in a, e.g., Adapt78 gene can be assayed in a biological sample. Such assays can be used for prognostic or predictive purpose to thereby prophylactically treat an individual prior to the onset of a disorder characterized by or associated with, e.g., Adapt78 protein, nucleic acid expression or activity.

Another aspect of the invention provides methods for determining, e.g., Adapt78 protein, multimer nucleic acid expression or, e.g., Adapt78 activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Diagnostic Assays

An exemplary method for detecting the presence or absence of, e.g., Adapt78 in a biological sample involves obtaining a biological sample from a test subject and contacting the, biological sample with a compound or an agent capable of detecting, e.g., Adapt78 protein, e.g., Adapt78 multimer or nucleic acid (e.g., mRNA, genomic DNA) that encodes, e.g., Adapt78 protein such that the presence of, e.g., Adapt78 is detected in the biological sample. An agent for detecting, e.g., Adapt78 mRNA or genomic DNA is a labeled nucleic acid probe capable of hybridizing to, e.g., Adapt78 mRNA or genomic DNA. The nucleic acid probe can be, for example, a full-length, e.g., Adapt78 nucleic acid or a portion thereof, such as an oligonucleotide of at least 15, 30, 50, 100, 250 or 500 nucleotides in length and sufficient to specifically hybridize under stringent conditions to, e.g., Adapt78 mRNA or genomic DNA.

An agent for detecting, e.g., Adapt78 protein or, e.g., Adapt78 multimer is an antibody capable of binding to, e.g., Adapt78 protein or, e.g., Adapt78 multimer, preferably an antibody with a detectable label. Antibodies can be polyclonal, or more preferably, monoclonal. An intact antibody, or a fragment thereof (e.g., F_{ab} or F_{(ab′)2}) can be used. “Labeled”, with regard to the probe or antibody, includes direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. “Biological sample” includes tissues, cells and biological fluids isolated from a subject, as well as tissues, cells and fluids present within a subject. That is, the detection method of the invention can be used to detect, e.g., Adapt78 mRNA, protein, or genomic DNA in a biological sample in vitro as well as in vivo. For example, in vitro techniques for detection of, e.g., Adapt78 mRNA include Northern hybridizations and in situ hybridizations. In vitro techniques for detection of, e.g., Adapt78 protein include enzyme linked immunosorbent assays (ELISAs), Western blots, immunoprecipitations and immunofluorescence. In vitro techniques for detection of, e.g., Adapt78 genomic DNA include Southern hybridizations. Furthermore, in vivo techniques for detection of, e.g., Adapt78 protein include introducing into a subject a labeled anti-Adapt78 antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting, e.g., Adapt78 protein, multimers, mRNA, or genomic DNA, such that the presence of, e.g., Adapt78 protein, multimers, mRNA or genomic DNA is detected in the biological sample, and comparing the presence of, e.g., Adapt78 protein, mRNA or genomic DNA in the control sample with the presence of, e.g., Adapt78 protein, multimers, mRNA or genomic DNA in the test sample.

The invention also encompasses kits for detecting the presence of, e.g., Adapt78 in a biological sample. For example, the kit can comprise: a labeled compound or agent capable of detecting, e.g., Adapt78 protein, multimer or mRNA in a biological sample; means for determining the amount of, e.g., Adapt78 in the sample; and means for comparing the amount of, e.g., Adapt78 in the sample with a standard. The compound or agent can be packaged in a suitable container. The kit can further comprise instructions for using the kit to detect, e.g., Adapt78 protein or nucleic acid.

Prognostic Assays

The diagnostic methods described herein can furthermore be utilized to identify subjects having or at risk of developing a disease or disorder associated with aberrant, e.g., Adapt78 expression or activity. For example, the assays described herein, such as the preceding diagnostic assays or the following assays, can be utilized to identify a subject having or at risk of developing a disorder associated with, e.g., Adapt78 protein, multimer, nucleic acid expression or activity such as cancer. Alternatively, the prognostic assays can be utilized to identify a subject having or at risk for developing a disease or disorder. Thus, the present invention provides a method for identifying a disease or disorder associated with aberrant, e.g., Adapt78 expression or activity in which a test sample is obtained from a subject and, e.g., Adapt78 protein or nucleic acid (e.g., mRNA, genomic DNA) is detected, wherein the presence of, e.g., Adapt78 protein or nucleic acid is diagnostic for a subject having or at risk of developing a disease or disorder associated with aberrant, e.g., Adapt78 expression or activity. “Test sample” includes a biological sample obtained from a subject of interest, e.g., a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant, e.g., Adapt78 expression or activity. Thus, the present invention provides methods for determining whether a subject can be effectively treated with an agent for a disorder associated with aberrant, e.g., Adapt78 expression or activity in which a test sample is obtained and, e.g., Adapt78 protein or nucleic acid is detected (e.g., wherein the presence of, e.g., Adapt78 protein or nucleic acid is diagnostic for a subject that can be administered the agent to treat a disorder associated with aberrant, e.g., Adapt78 expression or activity.) The methods of the invention can also be used to detect genetic lesions in a, e.g., Adapt78 gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a, e.g., Adapt78-protein, or the mis-expression of the, e.g., Adapt78 gene. For example, such genetic lesions can be detected by ascertaining the existence of at least one of (1) a deletion of one or more nucleotides from a, e.g., Adapt78 gene; (2) an addition of one or more nucleotides to a, e.g., Adapt78 gene; (3) a substitution of one or more nucleotides of a, e.g., Adapt78 gene, (4) a chromosomal rearrangement of a, e.g., Adapt78 gene; (5) an alteration in the level of a messenger RNA transcript of a, e.g., Adapt78 gene, (6) aberrant modification of a, e.g., Adapt78 gene, such as of the methylation pattern of the genomic DNA, (7) the presence of a non-wild type splicing pattern of a messenger RNA transcript of a, e.g., Adapt78 gene, (8) a non-wild type level of a, e.g., Adapt78-protein, (9) allelic loss of a, e.g., Adapt78 gene, and (10) inappropriate post-translational modification of a, e.g., Adapt78-protein. As described herein, there are a large number of assay techniques known in the art which can be used for detecting lesions in a, e.g., Adapt78 gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the, e.g., Adapt78-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a, e.g., Adapt78 gene under conditions such that hybridization and amplification of the, e.g., Adapt78 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a, e.g., Adapt78 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in, e.g., Adapt78 can be identified by hybridizing a sample and control nucleic acids, e.g., DNA or RNA, to high density arrays containing hundreds or thousands of oligonucleotides probes (Cronin et al. (1996) Mutation 7: 244-255; Kozal et al. (1996) Nature Medicine 2: 753-759). For example, genetic mutations in, e.g., Adapt78 can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the, e.g., Adapt78 gene and detect mutations by comparing the sequence of the sample, e.g., Adapt78 with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the, e.g., Adapt78 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type, e.g., Adapt78 sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in, e.g., Adapt78 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a, e.g., Adapt78 sequence, e.g., a wild-type, e.g., Adapt78 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in, e.g., Adapt78 genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control, e.g., Adapt78 nucleic acids will be denatured and allowed to re-anneal. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found (Saiki et al. (1986) Nature 324:163); Saiki et al. (1989) Proc Natl Acad. Sci USA 86:6230). Such allele-specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al (1992) Mol Cell Probes 6:1). It is anticipated that in certain embodiments amplification may also be performed using Taq ligase for amplification (Barany (1991) Proc Natl Acad Sci USA 88:189). In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a, e.g., Adapt78 gene.

Furthermore, any cell type or tissue, preferably peripheral blood leukocytes, in which, e.g., Adapt78 is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on, e.g., Adapt78 or, e.g., Adapt78 multimer activity (e.g., Adapt78 gene expression), as identified by a screening assay described herein can be administered to individuals to treat (prophylactically or therapeutically) disorders (e.g., cancer, ovulatory disorders, infertility or hypogonadism) associated with aberrant, e.g., Adapt78 activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of, e.g., Adapt78 protein, expression of, e.g., Adapt78 nucleic acid, or mutation content of, e.g., Adapt78 genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, Clin Exp Pharmacol Physiol, 1996, 23:983-985 and Linder, Clin Chem, 1997, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of, e.g., Adapt78 protein, e.g., Adapt78 multimer, expression of, e.g., Adapt78 nucleic acid, or mutation content of, e.g., Adapt78 genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a, e.g., Adapt78 modulator, such as a modulator identified by one of the exemplary screening assays described herein.

Peptide screening. Peptides and proteins generated, including those described in Examples 1-5, were screened for calcineurin activity modulation by preincubating each peptide-based composition with human calcineurin at 30° C., then adding the remaining components of the assay using the BIOMOL calcineurin assay kit and determining calcineurin phosphatase activity according to the manufacturer (BIOMOL Research Laboratories, Plymouth Meeting, Pa.). Those compositions that modulated calcineurin activity were chosen for further testing of their action on human cells in culture. These analyses consisted of exposing human cells such as IMR-90 fibroblasts, U251 astroglioma, MCF7 breast adenocarcinoma, HeLa epitheloid carcinoma, HL60 promyelocytes, and BEC(2)-M17 neuroblastoma cells and primary mouse cardiomyocytes cells to such compositions for various periods of time in cell culture, then measuring calcineurin activity in cell lysates. In the case of cardiomyocytes, the effect of each compound was also tested on the levels and activities of markers of cardiac hypertrophy after induction of hypertrophy by addition of various hypertrophic agonists.

Chemical compound screening. Selected chemical compounds were originally obtained from various chemical libraries for testing. Each stock chemical compound (and separately, control solvent) was diluted to an initial concentration of 50 μM and then preincubated at 30° C., in separate assays, with purified human Adapt78-FLAG or human Adapt78 protein, or the various Adapt78 peptides described above that were identified as SEQ ID NO:1 and SEQ ID NO:2. After subsequent preincubation with human calcineurin, the remaining calcineurin assay components were added for 15 minutes using the BIOMOL calcineurin assay kit (BIOMOL Research Laboratories, Plymouth Meeting, Pa.). Adapt78-FLAG was purified according to the FLAG manufacturer (Sigma) after expression in human cells in culture, and Adapt78 without the FLAG epitope tag was purified after expression in human cells in culture using a rabbit polyclonal antibody that we raised against Adapt78. These proteins were synthesized from pcDNA3.1 expression vectors (Invitrogen, Carlsbad, Calif.) containing each corresponding adapt78 cDNA according to the manufacturer. Compounds that either increased or decreased the inhibition of Adapt78 or Adapt78 peptides on calcineurin were chosen for further testing in whole cells. These analyses consisted of exposing human cells such as IMR-90 fibroblasts, U251 astroglioma, MCF7 breast adenocarcinoma, HeLa epitheloid carcinoma, HL60 promyelocytes, and BEC(2)-M17 neuroblastoma cells and primary mouse cardiomyocytes cells to such compounds and for various periods of time in cell culture, then measuring calcineurin activity in cell lysates. In the case of cardiomyocytes, the effect of each compound was also tested on the levels and activities of markers of cardiac hypertrophy after induction of hypertrophy by addition of various hypertrophic agonists.

The same approach was used to select potent calcineurin modulators using combinatorial chemicals. To obtain these, structural information (e.g., the importance of hydrophobic versus hydrophilic- and bulky-structures) from the peptide-based and chemical library-based modulators of calcineurin was combined and used to generate second and later generation combinatorial compounds with improved potency.

The invention includes modulators of calcineurin related to—and derived from—other members of the adapt78 gene family known as ZAKI-4/MCIP2, and DSCR1L2, which share a common highly homologous calcineurin inhibitory site. The above approaches and considerations would apply to these other adapt78 gene members, including all the variant forms (e.g., splice variants) of any member of this family.

Pharmaceutical composition. Any embodiments of the composition of the invention described herein may further comprise pharmaceutically acceptable carriers known in the art and can be formulated with the composition of the invention using methods known in the art. These include encapsulations for oral administration, nasal spray for nasal adminsitration, injection, and skin transdermal delivery.

The nucleic acid molecules, proteins, and antibodies (also referred to as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier.

“Pharmaceutically acceptable carrier” includes any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include water, saline, finger's solutions, dextrose solution, and 5% serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., protein, peptide or antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

To maximize efficient uptake of these compounds to humans and improve the specificity of target delivery, other embodiments of the composition of the invention described herein may further comprise structural modifications for intracellular localization (e.g., TAT peptide as described herein), tissue selectivity and intracellular localization and may include peptide-based vehicles containing multiple targeting signals, nanoparticles, and polysaccharide colloidal particles. See, e.g., Gariepy et al. Trends Biotechnol 19:21-28 (2001). This approach may also include viral vectors for delivering human DNA sequences containing human cDNAs, or selected portions of these cDNAs, to adapt78/DSCR1/MCIP1, ZAKI4/MCIP2, and DSCR1L2 to achieve the above desired therapeutic outcomes.

Transgenic mice. Transgenic mice overexpressing a human adapt78 transgene have been produced, as described in Example 6. These mice are useful in screening chemical compounds to identify those able to modulate the effect of Adapt78 protein (and the calcineurin modulators of the invention) on calcineurin in vivo. These mice are useful in studying, understanding and developing therapeutic treatments against adapt78- and calcineurin-related pathologies and abnormalities such as cardiac hypertrophy, memory loss, immune system dysfunction, developmental disabilities, etc. Furthermore, any cells and tissue derived from adapt78 transgenic mice, including fetuses, can be useful as well, such as for use and study in derived cell culture.

The host cells of the invention can also be used to produce nonhuman transgenic animals. For example, in one embodiment, a host cell of the invention is a fertilized oocyte or an embryonic stem cell into which Adapt78 protein-coding sequences have been introduced. Such host cells can then be used to create non-transgenic animals in which exogenous Adapt78 sequences have been introduced into their genome or homologous recombinant animals in which endogenous Adapt78 sequences have been altered. Such animals are useful for studying the function and/or activity of Adapt78 protein and for identifying and/or evaluating modulators of Adapt78 protein activity.

A transgenic animal of the invention can be created by introducing Adapt78-encoding nucleic acid into the male pronuclei of a fertilized oocyte, e.g., by microinjection, retroviral infection, and allowing the oocyte to develop in a pseudopregnant female foster animal. An adapt78 cDNA sequence can be introduced as a transgene into the genome of a non-animal. Alternatively, a nonhuman homolog of the adapt78 gene, such as a mouse adapt78 gene, can be isolated based on hybridization to the adapt78 cDNA and used as a transgene. Intronic sequences and polyadenylation signals can also be included in the transgene to increase the efficiency of expression of the transgene. A tissue-specific regulatory sequence(s) can be operably linked to the adapt78 transgene to direct expression of Adapt78 protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, in: Manipulating the Mouse Embryo, Cold Spring Harbor Laboratory Press, Cold Spring Harbor, N.Y. Similar methods are used for production of other transgenic animals. A transgenic founder animal can be identified based upon the presence of the adapt78 transgene in its genome and/or expression of adapt78 mRNA in tissues or cells of the animals. A transgenic founder animal can then be used to breed additional animals carrying the transgene. Moreover, transgenic animals carrying a transgene encoding Adapt78 can further be bred to other transgenic animals carrying other transgenes.

To create a homologous recombinant animal, a vector is prepared which contains at least a portion of an adapt78 gene into which a deletion, addition or substitution has been introduced to thereby alter, e.g., functionally disrupt, the adapt78 gene. The adapt78 gene can be a gene, but more preferably is a non-homolog of an adapt78 gene. For example, a mouse homolog of an adapt78 gene can be used to construct a homologous recombination vector suitable for altering an endogenous adapt78 gene in the mouse genome. In one embodiment, the vector is designed such that, upon homologous recombination, the endogenous adapt78 gene is functionally disrupted (i.e., no longer encodes a functional protein; also referred to as a “knock out” vector).

Alternatively, the vector can be designed such that, upon homologous recombination, the endogenous adapt78 gene is mutated or otherwise altered but still encodes functional protein (e.g., the upstream regulatory region can be altered to thereby alter the expression of the endogenous Adapt78 protein). In the homologous recombination vector, the altered portion of the adapt78 gene is flanked at its 5′ and 3′ ends by additional nucleic acid of the adapt78 gene to allow for homologous recombination to occur between the exogenous adapt78 gene carried by the vector and an endogenous adapt78 gene in an embryonic stem cell. The additional flanking adapt78 nucleic acid is of sufficient length for successful homologous recombination with the endogenous gene. Typically, several kilobases of flanking DNA (both at the 5′ and 3′ ends) are included in the vector. See, e.g., Thomas et al. (1987) Cell 51:503 for a description of homologous recombination vectors. The vector is introduced into an embryonic stem cell line (e.g., by electroporation) and cells in which the introduced adapt78 gene has homologously recombined with the endogenous adapt78 gene are selected (see e.g., Li et al. (1992) Cell 69:915).

The selected cells are then injected into a blastocyst of an animal (e.g., a mouse) to form aggregation chimeras. See, e.g., Bradley 1987, in: Teratocarcinomas and Embryonic Stem Cells: A Practical Approach, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-human animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to a pseudopregnant female foster animal. The offspring borne of this female foster animal will be a clone of the animal from which the cell, e.g., the somatic cell, is isolated.

### EXAMPLE 1

Peptide synthesis. All peptides were synthesized using FMOC solid phase peptide synthesis on a Pioneer Peptide Synthesis System (PerSeptive Biosystems, Inc.). Briefly, peptides were assembled on a solid support (FMOC-Polyethylene-Graft Polystyrene Support) from the C-terminus, reacting the deblocked amino (N)-terminus of support-bound amino acid with the activated carboxyl (C)-terminus of the incoming amino acid to form an amide bond. Amino acids used in the synthesis have their N^{a}-amino group protected by the 9-fluorenylmethoxycarbonyl (FMOC) group, which is removed by piperidine at the end of each cycle in the synthesis. The carboxyl-group of the amino acid was activated with HATU [O-(7-azabenzotriazol-1-yl)-1,1,3,3-tetramethyluronium hexafluorophosphate] obtained from PerSeptive. Side-chain protecting groups of amino acids were removed by trifluoroacetic acid (TFA) after peptide synthesis. The specific amino acid derivatives, supports, and reagents used in the synthesis were purchased from PerSeptive or Nova Biochem.

After synthesis was completed, the resin was washed three times with 100% propanol and the cleavage reaction was initiated by incubating the resin in 1 Oml TFA/thioanisole/anisole/1,2-ethanedithiol (90:5:2:3) (or other appropriate cleaving reagent) per 0.5 g resin for 5 hours. The cleavage reaction mixture was filtered using a sintered glass funnel to separate the solid resin from the peptide solution. Filtrate volume was reduced to 1 ml by evaporation facilitated with a gentle stream of air, and the peptides were precipitated by addition of 15 ml dry ice-chilled ethyl ether. The peptides were allowed to settle for five minutes at −80° C., and the supernatant was aspirated. The peptides were then washed twice in similar manner with 15 ml of ethyl ether. After three further washings with 15 ml of ethyl acetate:diethylether (1.5:1, room temperature), the peptides were dissolved in deionized water, purified by reverse phase HPLC, lyophilized and stored at −20° C.

### EXAMPLE 2

Cyclization of Peptides. Backbone atom peptide cyclization was accomplished using methods described by Kates et al. Anal Biochem 212:303-310 (1993). Briefly, N-alpha FMOC-L-glutamic acid-alpha-allyl ester at the C-terminus of a synthetic peptide was coupled to the resin via the gamma carboxylic acid. Removal of the FMOC allows the remaining amino acids to be incorporated sequentially into the growing peptide. A free alpha-carboxyl group was then generated upon removal of allyl group. This alpha-carboxyl group was then coupled to the free N-terminal residue of the peptide (while on the resin) in order to generate the cyclic peptide, which was then removed from the resin in such a way to yield the gamma-carboxamido derivative (i.e., Q). The cyclic peptide was then purified and characterized as described in Example 3.

### EXAMPLE 3

Peptide Purification. Peptide purification was done using a Waters Delta-Pak C₁₈ reverse phase column (19 mm×30 cm) with a pore diameter of 300 Å. The system used for purification included a Waters model 650E liquid chromatography system equipped with a model 486 adjustable absorbance detector and a model 600E controller. Peptides were monitored at 230 nm (assuming no aromatic chromophores are present). The column was operated from 0 to 4 min in 0.1% trifluoroacetic acid aqueous mobile phase, followed by a linear gradient to 60% acetonitrile, at a flow rate of 7 ml/min. Fractions containing pure peptide (>98% purity) were pooled together and lyophilized. Subsequently, peptides were re-lyophilized (removal of TFA) and stored at −20° C. under inert gas environment.

### EXAMPLE 4

Peptide characterization and design. Amino acid analyses of all peptides was performed using the Waters AccQ-Tag amino acid analysis system. See, e.g., Strydom et al. Anal Biochem 222:19-28 (1994). Peptides were analyzed by mass spectrometry using standard α-cyano-4-hydroxysinnipinic acid and sinnipinic acid matrices. Integrity of cyclized peptides was further validated using the Kaiser test to ensure absence of free terminal amino group.

a. Decreased Size. Progressively smaller analogs were made from the peptide shown in SEQ ID NO:1 to identify the minimal active sequence. The approach is similar to the series of parsing efforts, noted above, that led from intact AFP to an octapeptide.

b. Improved Solubility and Stability Enhancement. Where solubility is a problem, we incorporated polar or charged residues for non-essential (or at least non-conserved) hydrophobic residues. Judicial replacement of amino acids can lead to an improvement in activity in some cases (and to loss of activity in many cases, which helps to identify ‘essential’ residues). Choices for such substitutions comes with experience and with use of the molecular modeling programs, considerations of homology between species, and examination of crystal structures when available. Further, it was also desirable to incorporate D-amino acid(s) to diminish proteolysis. Thus, we have substituted at several positions in the peptide, using mostly natural L-amino acids, some D-amino acids and some of the commercially available structural analogs of amino acids (e.g., hydroxyproline for proline).

c. Cyclization. Conformational constraint of peptides can provide additional stability and enhanced bioactivity. By decreasing the number of conformers available to a peptide, it can sometimes be locked in to an active shape. Cyclization was done during synthesis (while the given Adapt78 peptide was on the resin). This approach generated a C-terminal carboxyl-to-N-terminal amine nitrogen peptide bond. Cyclization was also done utilizing side chain functional groups as described by Spinella et al., Proc Natl Acad Sci USA 88:7443-7446 (1991).

d. Final optimization. After generating the shortest active peptides (cyclic and linear), other analogs were generated to increase activity. Combinatorial replacement of non-essential amino acids (one at a time) was performed to screen several hundred analogs, and the identification of potent analogs was followed directed, single replacements at that position in the optimal peptide.

### EXAMPLE 5

Adapt78-TAT fusion protein. Adapt78-TAT fusion protein was constructed by adding the 10 amino acid HIV TAT protein transduction domain (see, e.g., Vives et al., J Biol Chem 272:16010-16017 (1997); Bonny et al., Diabetes 50:77-82 (2001); and Nagahara et al. Nat Med 4:1449-1452 (1998)) followed by two glycine residues, GRKKRRQRRR-GG (SEQ ID NO:5), in frame to the amino end of the open reading frame for human adapt78, with and without the addition of two FLAG epitope tags (Sigma Chemical Company, St. Louis, Mo.) in frame at the very C-terminus. The DNA was propagated and protein expressed and purified as described by the manufacturer (Sigma) or as described above.

### EXAMPLE 6

Adapt78 transgenic mice. Human adapt78-containing BAC clone was purified with Nucleobond AX, resuspended in nuclease-free water, and purity assessed by pulse field gel electrophoresis. The DNA was then further diluted in microinjection buffer. This DNA was microinjected into the pronuclei of C57BL/6 inbred mouse eggs and surviving eggs reimplanted into pseudopregnant recipient mice. Founder transgenic mice were identified by PCR analysis of tail DNA using primers specific to human adapt78. Identified founders were then bred with “control” litter mates to establish F1 transgenic lines, then intermated to generate F2 homozygote, heterozygote and wild-type mice. The adapt78 transgenic mice were also interbred with SJL mice to generate C57BL/6×SJL hybrid adapt78 transgenic mice. Transgenic fetuses are also included under our definition of adapt78 transgenic mice and are derived as described above, also with utility in a study, understanding, diagnosis, and therapeutic treatment of adapt78- and calcineurin-based pathologies.

### EXAMPLE 7

MATERIAL AND METHODS: Cell culture and treatment conditions. Chinese hamster HA-1 fibroblasts were maintained in Eagle's minimal essential medium supplemented with 8% heat inactivated fetal bovine serum, penicillin (100 units/ml) and streptomycin (100μg/ml). Cultures were grown in a humidified incubator atmosphere of 95% air and 5% CO₂ at 37□C.

Stable transfection. Full-length cDNA to hamster adapt78 was inserted into the mammalian expression vector pcDNA3.1 according to the manufacturer (Invitrogen, LaJolla, Calif.). This recombinant, as well as pcDNA3.1 vector controls, were purified using Qiagen Maxiprep columns according to the manufacturer (Qiagen Corporation, Valencia, Calif.). Final DNA was resuspended in autoclaved distilled, deionized water and the DNA identities were confirmed by restriction mapping and sequencing. Each DNA preparation was then transfected into HA-1 cells using BES-mediated calcium phosphate as described in Sambrook et al., Molecular Cloning: A laboratory Manual, Cold Spring Harbor Laboratory Press, Cold Spring Harbor, N.Y. (1989). G418-resistant HA-1 colonies were then selected and screened for successful adapt78 stable integration and overexpression using standard technique of Crawford et al., Methods in Enzymology 234, 175-217 (1994).

Stress analyses. Control cells and lines 7, 10, and 15 were grown to log phase and equal confluency (75%). At this time, the cells were exposed to each stress agent and cytotoxicity measured. For the oxidative stress studies, a stock solution of hydrogen peroxide (30% w/w) was diluted in phosphate-buffered saline just prior to cell exposure. At the time of treatment, the HA-1 cultures were taken from the incubator and freshly diluted-peroxide added to the culture dishes. The cells were then returned to the incubator for the appropriate times.

Stable adapt78-overexpressing, vector control and non-transfected control HA-1 cells were exposed to 4 μg/ml calcium ionophore A23187 or 5 mM for 60 minutes (A23187) or 90 minutes (hydrogen peroxide). Cytotoxicity was then determined by propidium iodide uptake using flow cytometry as follows. After treatment, monolayers were rinsed with PBS containing 3 mM EDTA, then covered with the same for 5 minutes at room temperature before suspending cells by gentle trituration. Cytotoxicity was then measured using propidium iodide and the stained cells analyzed by flow cytometry using a Becton Dickinson FAC Scan. The ModFit analytical software program was used to quantify the percentage of cytotoxicity.

Growth analyses. The above transfectants used for cytoprotection analysis were employed. Cells were trypsinized and plated at 10,000 cells per 60 mm plate and cells counted using a hematocytometer at multiple time points.

Cell cycle analysis. Cell cycle analysis was performed using flow cytometry. Randomly cycling cells were trypsinized and fixed with 90% ice cold methanol. Cells were then centrifuged and the methanol supernatant removed. Cells were then resuspended in PBS and RNase added to a final concentration of 0.5 mg/ml. After a 30 minute incubation at room temperature, propidium iodide was added and cell cycle distribution analyzed on a Becton Dickinson flow cytometer. The ModFitLT flow cytometry computer program, version 2.0, was used to analyze data.

Adapt78 antibody. Using the predicted open reading frame sequence of the predicted Adapt78 protein as a reference, we chose an 8 amino acid stretch near the C-terminus that was predicted to be exposed. This peptide (EMERMPKP (SEQ ID NO: 6)) was conjugated to keyhole limpet hemacyanin, injected into rabbits, and polyclonal antibody raised against this epitope by SynPep, Co. (Dublin, Calif.). The final batch of serum was affinity purified on a column with the Adapt78 peptide covalently attached. Final dilutions were routinely 1:500 for Western blot analysis and 1:50 or 1:100 for immunohistochemistry.

Protein gel analyses. Monolayer cells were washed in PBS and cells lysed with 1 ml of 15 mM CHAPS detergent, 1 mM EDTA, 20 mM Tris-HCl, pH 7.5, and protease inhibitor cocktail (Boehringer Mannheim, Indianapolis, Ind.) per 10⁷ cells. The lysates were then mixed with an equal volume of 2×Laemmli sample buffer (54) and 20 μg (per lane) electrophoresed on a 15% SDS-polyacrylamide gel. After electroblotting to Nitrocellulose membrane according to the manufacturer (Schleicher and Schuell, Keene, N.H.), Adapt78 signal was detected by hybridization to the above Adapt78 antibody followed by Super signal (Pierce Chemicals, Rockford, Ill.) and the image capured by phophoimaging using the Storm 860 phosphoimager and ImageQuant software (Molecular Dynamics, Riverdale, Calif.). For one lane, an excess of Adapt78 peptide was preincubated with antibody for one hour prior to antibody hybridization.

In vitro transcription and translation. The TnT wheat germ kit was used to transcribe and translate hamster adapt78 cDNA in the pcDNA3.1 vector according to the manufacturer (Promega Biotec, Madison, Wis.). In brief, 1 μg of the above construct, pcDNA3.1-vector alone, and no DNA, were transcribed and translated and the reaction products electrophoresed through a 15% polyacrylamide/SDS gel. After transfer to nitrocellulose membrane, the Adapt78 signal was determined using antibody as described above except that Pierce Super Signal Ultra was used.

Immunohistochemistry. HA-1 cells were plated on culture dishes containing coverslips. Twenty four hours later, the cells were fixed to the coverslips by incubation for 10 minutes in 3.7% paraformaldehyde dissolved in phosphate-buffered saline (PBS). The cells were permeabilized for five minutes using a 0.1% Triton X-100 solution in PBS, aldehydes blocked with a 100 mM glycine-PBS solution, and the coverslips washed twice with PBS. Samples were then incubated with Adapt78 antibody in 5% goat serum overnight at 4□ C. The samples were washed for five minutes with PBS, and FITC-conjugated goat anti-rabbit IgG secondary antibody (1:75) added for 30 minutes at room temperature in the dark. The coverslips were washed with PBS for 5 minutes and then mounted on glass slides using Vectorshield (Vector Laboratories, Burlingame, Calif.) as a mounting medium. The coverslips were fixed in place using clear nail polish and visualized on a Nikon fluorescence microscope at 40× and 100×. Pictures were taken using Kodak TMAX 400 film.

Results: Adaptive response. In response to a moderate “pretreatment” dose of hydrogen peroxide (160 μM), HA-1 cells mount a protective response that renders them more resistant to subsequent exposure to a higher dose of peroxide. As previously reported, exposure of HA-1 cells to this pretreatment concentration of hydrogen peroxide leads to stress protection and growth arrest.

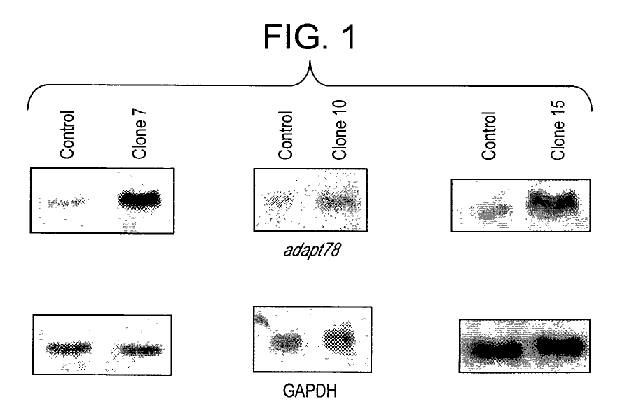

Stable transfection. Based on the adaptive response studies, we decided to assess the role, if any, of each adapt genes in cytoprotection and growth arrest, beginning with adapt78. Stable transfection of HA-1 cells with hamster adapt78 cDNA generated several sublines (designated 7, 10 and 15) overexpressing adapt78 mRNA (FIG. 1). mRNA expression analysis revealed a modest average level of adapt78 overexpression in these transfectants (3.1-, 1.5- and 2.1-fold over control levels, respectively, for clones 7, 10 and 15). No lines were isolated with a greater overexpression than these, even after selecting and screening over 47 G418-resistant HA-1 colonies after adapt78 stable transfection. This suggests that adapt78 overexpression has a profound effect on HA-1 cells that is only tolerated up to a certain (i.e., modest) level.

adapt78 is cytoprotective. Since we originally identified adapt78 using an adaptive response model system where cellular cytoprotection to hydrogen peroxide and growth arrest were found to be correlated with adapt78 induction, we decided to assess the direct effect of adapt78 overexpression on cytoprotection. In addition, we have described adapt78 as a putative new member of the grp family of stress genes, and there is evidence that grp78 is cytoprotective. Although our original analyses were performed with hydrogen peroxide, we have since found that calcium ionophore A23187 is a stronger inducer of adapt78 mRNA. This observation, combined with the induction of adapt78 mRNA by other agents that raise intracellular calcium (thapsigargin, cyclopiazonic acid and peroxide) led us to assess adapt78 cytoprotection using high concentrations of both A23187 and peroxide.

Control cells and lines 7, 10, and 15 were grown to log phase and equal confluency (75%), then treated with 4 μg/ml A23187 and separately, 5 mM hydrogen peroxide. Cytotoxicity was then assessed by propidium iodide uptake using flow cytometry. Fold protection was calculated as the percent viability of cells compared with untransfected after subtracting out background (cells treated with solvent only). The average of untransfected percent viability was arbitrarily set at 1.0. As shown in Table 1, below, a strong inhibition of cytotoxicity was observed in the adapt78 overexpressing cells as compared with control. This was observed for both calcium and hydrogen peroxide stress. Thus, adapt78 induces a strong cytoprotection in HA-1 cells.

TABLE 1
The cytotoxic effect of A23187 and
hydrogen peroxide on stable transfectants

| | | | | Fold |
|---|---|---|---|---|
| | | | Percent | protection +/− |
| | | | cytotoxicity | 1 S.D. |
| HA-1 cell type | A23187 | Peroxide | A23187 | Peroxide |
| Untransfected | 48.2 | 59.9 | 1.0 +/− 0.4 | 1.0 +/− 0.3 |
| Vector transfected | 44.0 | 59.2 | 1.1 +/− 0.3 | 1.0 +/− 0.4 |
| Clone 7 | 10.1 | N.D. | 4.8 +/− 0.5 | N.D. |
| Clone 10 | 11.7 | 16.4 | 4.1 +/− 1.0 | 3.7 +/− 0.6 |
| Clone 15 | 11.8 | 14.2 | 4.1 +/− 0.3 | 4.2 +/− 0.6 |
| Note. | | | | |
| Control untransfected cells, pcDNA3.1 vector-only transfected cells (vector control), and adapt78 overexpressing lines 7, 10, and 15 were grown to log phase and equal confluency (75%), then treated with 4 μg/ml A23187 and separately, 5 mM hydrogen peroxide. Cytotoxicity was then assessed by propidium iodide uptake using flow cytometry. Fold protection was calculated as the % viability of cells compared with Untransfected. The average of Untransfected % viability was arbitrarily set | | | | |
| # at 1.0. S.D., standard deviation (n = 4). | | | | |

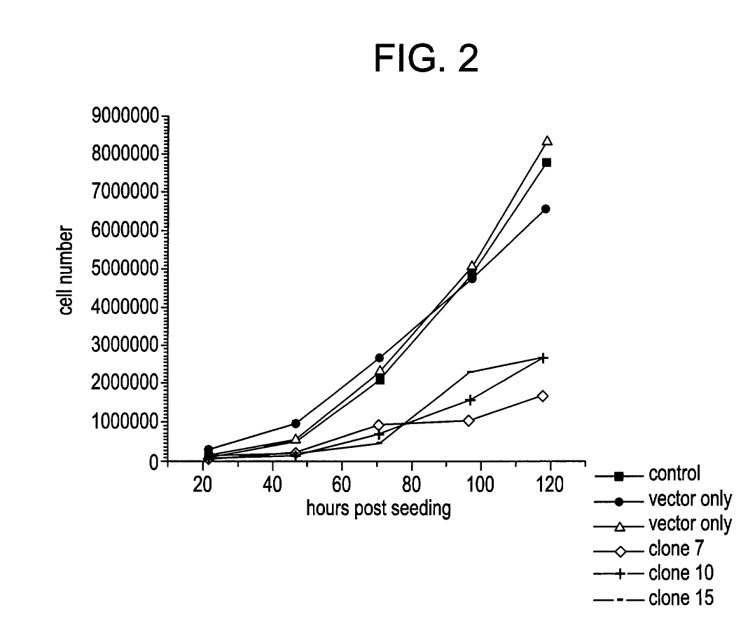

adapt78 is associated with growth suppression. When HA-1 cells exposed to hydrogen peroxide for 5 hours were plated at low density, then stained 7 days later with Giemsa, significant decreases in colony number were observed in the peroxide-treated cell populations as compared with control: 79.5% and 97.8%, respectively, for 160 and 400 μM hydrogen peroxide (62). At 160 μM, no associated cytotoxicity was observed using trypan blue exclusion. Furthermore, we observed significant growth arrest in our original adaptive response model system that correlated with stress-induction of adapt78 mRNA levels. Studies were therefore undertaken to test the direct effect of adapt78 on growth suppression. For each cell line, aliquots were plated at 10,000 cells per 60 mm plate and cells counted at multiple time points. Strong growth suppression was observed for all the adapt78 overexpressors as compared with controls (FIG. 2). This analysis was performed five times, each with the same strong growth suppression. The strongest growth suppression was observed in clone 7, which also exhibited the highest overexpression (3.1-fold and 3.2 fold, respectively, with respect to mRNA and protein). The correlation of modest adapt78 overexpression with strong growth arrest indicates that adapt78 is a strong growth suppressor. This growth arrest effect may also explain our inability to obtain large-fold adapt78 overexpressing clones as discussed above. Strong adapt78 overexpression most likely would suppress colony growth too much for subsequent colony isolation.

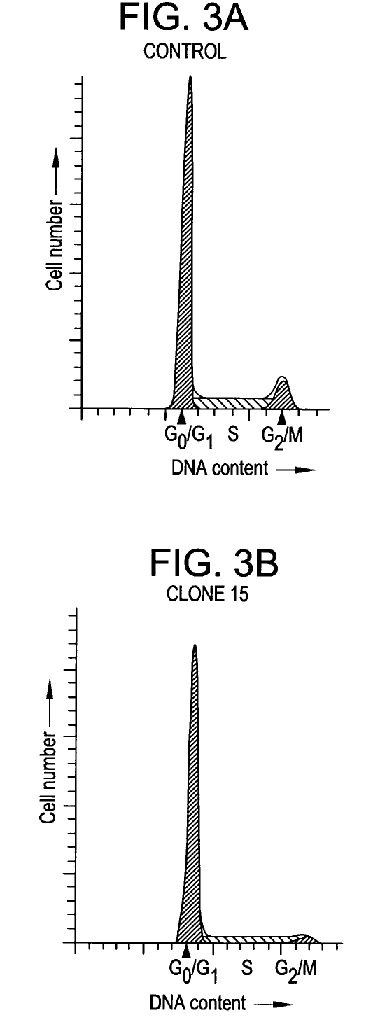

Growth arrest occurs during G₁ phase. To determine the stage of the cell cycle responsible for adapt78-induced growth suppression, we chose clone 15, which overexpresses adapt78 at an intermediate level (2.1-fold) among the three clones, for flow cytometry analysis. Clone 15 and vector control were grown to log phase, fixed, and cell cycle distribution assessed by flow cytometry following RNase treatment and propidium iodide staining. The ModFit analytical software program was used to determine the percentage of cells in different stages of the cells cycle. These stages were divided into G₀/G₁-, S-, and G₂/M-phase. As can be seen from FIG. 3, a significant suppression of the S-phase and G₂/M phase signals is observed in clone 15 cells with a concomitant increase in G₀/G₁-phase cells, as compared with control. For two analyses, a 32% decrease of S-phase and a 35% decrease of G₂/M phase with a concomitant increase in the G₀/G₁ population was observed in clone 15 as compared with controls. We therefore conclude that adapt78 overexpression induces growth suppression in G₁ phase.

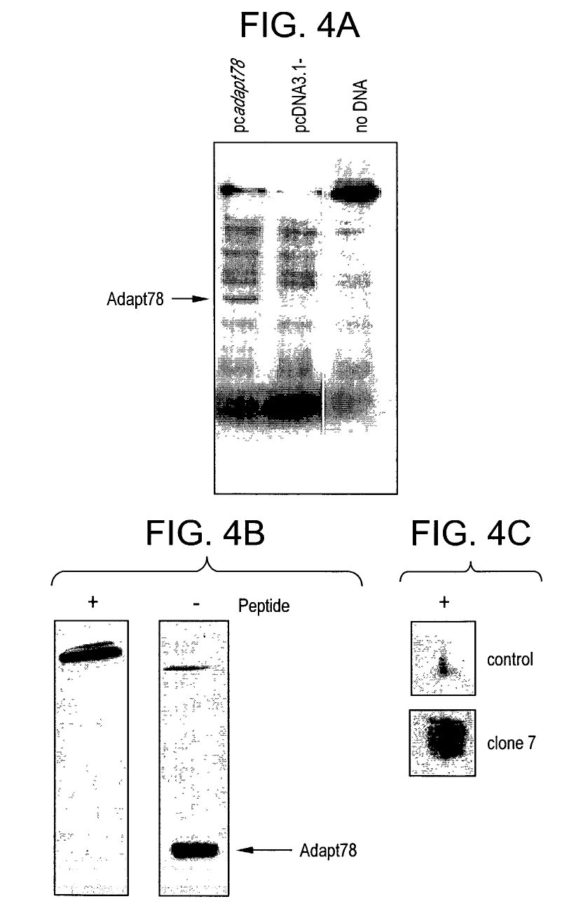

Adapt78 protein analyses. To study adapt78 at the level of its protein product, we raised an antibody against an 8-mer peptide encoded for by the C-terminal region of Adapt78. In vitro transcription and translation, followed by Western blot analysis and a sensitive detection method (Pierce Super Signal Ultra) revealed that the Adapt78 antibody bound to in vitro transcription translation-generated Adapt78 protein (FIG. 4A), and that its binding to HA-1 cell lysate was inhibited by an excess of the Adapt78 eight amino acid epitope peptide (FIG. 4B). Thus, the obtained antibody (anti78) is specific to Adapt78. This antibody also binds to Adapt78 that is overexpressed in stable transfectants as is shown for clone 7 in FIG. 4C.

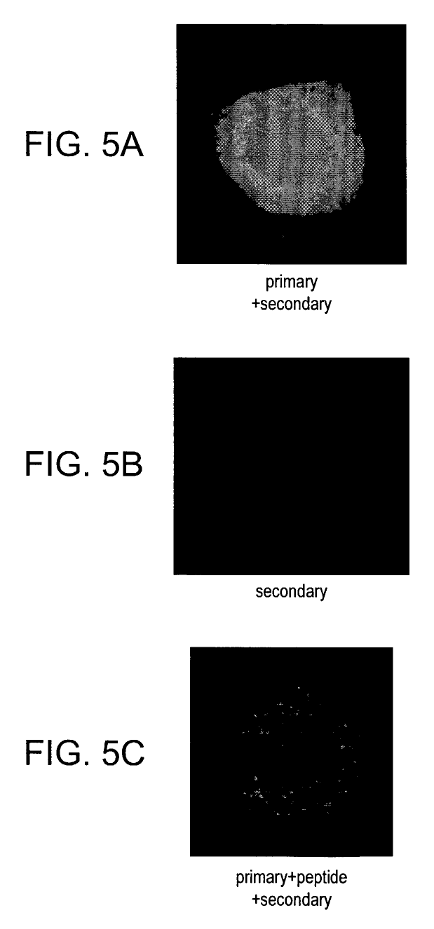

Adapt78 is predominantly perinuclear. We assessed the intracellular localization of Adapt78 on fixed and permeabilized HA-1 cells using the Adapt78 antibody. These cells were incubated with this primary antibody followed by FITC-conjugated goat anti-rabbit IgG and subsequent fluorescent microscopy. As controls, cells were incubated with either secondary antibody alone or with anti78 preabsorbed with Adapt78 peptide followed by secondary antibody. Subtracting out the secondary antibody-only staining reveals that a significant amount of Adapt78 protein localizes in the perinucleus. Significant cytoplasmic staining is also present (FIG. 5). The perinuclear staining results are similar to that previously reported for Grp78, a resident endoplasmic reticulum protein and a major stress protein. These results suggest that Adapt78 protein acts at the endoplasmic reticulum and cytoplasm to effect cytoprotection and growth arrest.

Equivalents

Those skilled in the art will recognize, or be able to ascertain using no more than routine experimentation, numerous equivalents to the specific procedures described herein. Such equivalents are considered to be within the scope of the present invention and are covered by the following claims. Various substitutions, alterations, and modifications may be made to the invention without departing from the spirit and scope of the invention as defined by the claims. Other aspects, advantages, and modifications are within the scope of the invention. The contents of all references, issued patents, and published patent applications cited throughout this application are hereby incorporated by reference. The appropriate components, processes, and methods of those patents, applications and other documents may be selected for the present invention and embodiments thereof.

References

1. Lai,M. M., P. E.Burnett, H.Wolosker, S.Blackshaw and S. H.Snyder. 1998. Cain, a novel physiologic protein inhibitor of calcineurin. J Biol Chem 273:18325-18331.

2. Molkentin,J. D., J. R.Lu, C. L.Antos, B.Markham, J.Richardson, J.Robbins, S. R.Grant and E. N.Olson. 1998. A calcineurin-dependent transcriptional pathway for cardiac hypertrophy. Cell 93:215-228.

3. Rao,A., C.Luo and P. G.Hogan. 1997. Transcription factors of the NFAT family: regulation and function. [Review] [220 refs]. Annu Rev Immunol 15:707-747.

4. Graef,I. A., P. G.Mermelstein, K.Stankunas, J. R.Neilson, K.Deisseroth, R. W.Tsien and G. R.Crabtree. 1999. L-type calcium channels and GSK-3 regulate the activity of NF-ATc4 in hippocampal neurons. Nature 401:703-708.

5. Naciff,J. M., K. L.King and J. R.Dedman. 2000. Targeted neutralization of calcineurin, by expression of an inhibitor peptide under the control of a cholinergic specific promoter in PC12 cells, promotes neurite outgrowth in the presence of NGF. Metab Brain Dis 15:65-81.

6. Ripka,A. S. and D. H.Rich. 1998. Peptidomimetic design. [Review] [65 refs]. Curr Opin Chem Biol 2:441-452.

7. Rusnak,F. and P.Mertz. 2000. Calcineurin: form and function. [Review] [487 refs]. Physiol Rev 80:1483-1521.

8. Kingsbury,T. J. and K. W.Cunningham. 2000. A conserved family of calcineurin regulators. Genes Dev 14:1595-1604.

9. Nichols,R. A., G. R.Suplick and J. M.Brown. 1994. Calcineurin-mediated protein dephosphorylation in brain nerve terminals regulates the release of glutamate. J Biol Chem 269:23817-23823.

10. Sigal,N. H., F.Dumont, P.Durette, J. J.Siekierka, L.Peterson, D. H.Rich, B. E.Dunlap, M. J.Staruch, M. R.Melino and S. L.Koprak. 1991. Is cyclophilin involved in the immunosuppressive and nephrotoxic mechanism of action of cyclosporin A? J Exp Med 173 :619-628.

11. Gorlach,J., D. S.Fox, N. S.Cutler, G. M.Cox, J. R.Perfect and J.Heitman. 2000. Identification and characterization of a highly conserved calcineurin binding protein, CBP1/calcipressin, in Cryptococcus neoformans. EMBO J 19:3618-3629.

12. Baksh,S. and S. J.Burakoff. 2000. The role of calcineurin in lymphocyte activation. Semin Immunol 12:405-415.

13. Fuentes,J. J., L.Genesca, T. J.Kingsbury, K. W.Cunningham, M.Perez-Riba, X.Estivill and L. S.de la. 2000. DSCR1, overexpressed in Down Syndrome, is an inhibitor of calcineurin-mediated signaling pathways. Hum Mol Genet 9:1681-1690.

14. Sussman,M. A., H. W.Lim, N.Gude, T.Taigen, E. N.Olson, J.Robbins, M. C.Colbert, A.Gualberto, D. F.Wieczorek and J. D.Molkentin. 1998. Prevention of cardiac hypertrophy in mice by calcineurin inhibition [see comments]. Science 281:1690-1693.

15. Taigen,T., L. J.De Windt, H. W.Lim and J. D.Molkentin. 2000. Targeted inhibition of calcineurin prevents agonist-induced cardiomyocyte hypertrophy. Proc Natl Acad Sci USA 97:1196-1201.

16. Zhang,B. W., G.Zimmer, J.Chen, D.Ladd, E.Li, F. W.Alt, G.Wiederrecht, J.Cryan, E. A.O'Neill, C. E.Seidman, A. K.Abbas and J. G.Seidman. 1996. T cell responses in calcineurin A alpha-deficient mice. J Exp Med 183:413-420.

17. Kayyali,U. S., W.Zhang, A. G.Yee, J. G.Seidman and H.Potter. 1997. Cytoskeletal changes in the brains of mice lacking calcineurin A alpha. J Neurochem 68:1668-1678.

18. Zhuo,M., W.Zhang, H.Son, I.Mansuy, R. A.Sobel, J.Seidman and E. R.Kandel. 1999. A selective role of calcineurin aalpha in synaptic depotentiation in hippocampus. Proc Natl Acad Sci USA 96:4650-4655.

19. Shibasaki,F. and F.McKeon. 1995. Calcineurin functions in Ca(2+)-activated cell death in mammalian cells. J Cell Biol 131:735-743.

20. Rothermel,B., R. B.Vega, J.Yang, H.Wu, R.Bassel-Duby and R. S.Williams. 2000. A protein encoded within the Down Syndrome critical region is enriched in striated muscles and inhibits calcineurin signaling. J Biol Chem 275:8719-8725.

21. Crawford,D. R., K. P.Leahy, N.Abramova, L.Lan, Y.Wang and K. J.Davies. 1997. Hamster adapt78 mRNA is a Down Syndrome critical region homolog that is inducible by oxidative stress. Arch Biochem Biophys 342:6-12.

22. Leahy,K. P., K .J.Davies, M.Dull, J. J.Kort, K. W.Lawrence and D. R.Crawford. 1999. adapt78, a stress-inducible mRNA, is related to the glucose-regulated protein family of genes. Arch Biochem Biophys 368:67-74.

23. Leahy,K. P. and D. R.Crawford. 2000. adapt78 protects cells against stress damage and suppresses cell growth. Arch Biochem Biophys 379:221-228.

24. Festin,S. M., J. A.Bennett, P. W.Fletcher, H. I.Jacobson, D. D.Shaye and T. T.Andersen. 1999. The recombinant third domain of human alpha-fetoprotein retains the antiestrotrophic activity found in the full-length molecule. Biochim Biophys Acta 1427:307-314.

25. Mesfin,F. B., J. A.Bennett, H. I.Jacobson, S.Zhu and T. T.Andersen. 2000. Alpha-fetoprotein-derived antiestrotrophic octapeptide. Biochim Biophys Acta 1501:33-43.

26. Leach,A. R. and M. M.Hann. 2000. The in silico world of virtual libraries. Drug Discov Today 5:326-336.

27. Maffla,A. M., I.Kariv and K. R.Oldenburg. 1999. Miniaturization of a Mammalian Cell-Based Assay: Luciferase Reporter Gene Readout in a 3 Microliter 1536-Well Plate. J Biomol Screen 4:137-142.

28. Deadman,J. 2000. Proteinase inhibitors and activators strategic targets for therapeutic intervention. J Pept Sci 6:421-431.

29. Forlenza,O. V., J. M.Spink, R.Dayanandan, B. H.Anderton, O. F.Olesen and S.Lovestone. 2000. Muscarinic agonists reduce tau phosphorylation in non-neuronal cells via GSK-3beta inhibition and in neurons. J Neural Transm 107:1201-1212.

30. Garver,T. D., R. L.Kincaid, R. A.Conn and M. L.Billingsley. 1999. Reduction of calcineurin activity in brain by antisense oligonucleotides leads to persistent phosphorylation of tau protein at Thr181 and Thr231. Mol Pharmacol 55:632-641.

31. Ladner,C. J., J.Czech, J.Maurice, S. A.Lorens and J. M.Lee. 1996. Reduction of calcineurin enzymatic activity in Alzheimer's disease: correlation with neuropathologic changes. J Neuropathol Exp Neurol 55:924-931.

32. Crawford,D. R. and K. J.Davies. 1994. Adaptive response and oxidative stress. [Review] [46 refs]. Environ Health Perspect 102:Suppl-8.

33. Epstein,C. J., J. R.Korenberg, G.Anneren, S. E.Antonarakis, S. Ayme, E.Courchesne, L. B.Epstein, A.Fowler, Y.Groner and J. L.Huret. 1991. Protocols to establish genotype-phenotype correlations in Down Syndrome. Am J Hum Genet 49:207-235.

34. Wang,W., R. T.Borchardt and B.Wang. 2000. Orally active peptidomimetic RGD analogs that are glycoprotein IIb/IIIa antagonists. [Review] [112 refs]. Curr Med Chem 7:437-453.

35. Cirillo,R., M.Astolfi, B.Conte, G.Lopez, M.Parlani, R.Terracciano, C. I.Fincham and S.Manzini. 1998. Pharmacology of the peptidomimetic, MEN 11149, a new potent, selective and orally effective tachykinin NK1 receptor antagonist. Eur J Pharmacol 341:201-209.

36. Kamm,W., P.Raddatz, J.Gante and T.Kissel. 1999. Prodrug approach for alphaIIbbeta3-peptidomimetic antagonists to enhance their transport in monolayers of a human intestinal cell line (Caco-2): comparison of in vitro and in vivo data. Pharm Res 16:1527-1533.

37. Saitoh,H. and B. J.Aungst. 1999. Improvement of the intestinal absorption of a peptidomimetic, boronic acid thrombin inhibitor possibly utilizing the oligopeptide transporter. Pharm Res 16:1786-1789.

38. Lark,M. W., G. B.Stroup, S. M.Hwang, I. E.James, D. J.Rieman, F. H.Drake, J. N.Bradbeer, A.Mathur, K. F.Erhard, K. A.Newlander, S. T.Ross, K. L.Salyers, B. R.Smith, W. H.Miller, W. F.Huffman and M.Gowen. 1999. Design and characterization of orally active Arg-Gly-Asp peptidomimetic vitronectin receptor antagonist SB 265123 for prevention of bone loss in osteoporosis. J Pharmacol Exp Ther 291:612-617.

39. O'Connor,S. J., K. J.Barr, L.Wang, B. K.Sorensen, A. S.Tasker, H.Sham, S. C.Ng, J.Cohen, E.Devine, S.Cherian, B.Saeed, H.Zhang, J. Y.Lee, R.Warner, S.Tahir, P.Kovar, P.Ewing, J.Alder, M.Mitten, J.Leal, K.Marsh, J.Bauch, D. J.Hoffman, S. M.Sebti and S. H.Rosenberg. 1999. Second-generation peptidomimetic inhibitors of protein farnesyltransferase demonstrating improved cellular potency and significant in vivo efficacy. J Med Chem 42:3701-3710.

40. Sanderson,P. E., T. A.Lyle, K. J.Cutrona, D. L.Dyer, B. D.Dorsey, C. M.McDonough, A. M.Naylor-Olsen, I. W.Chen, Z.Chen, J. J.Cook, C. M.Cooper, S. J.Gardell, T. R.Hare, J. A.Krueger, S. D.Lewis, J. H.Lin, B. J.Lucas, Jr., E. A.Lyle, J. J.Lynch, Jr., M. T.Stranieri, K.Vastag, Y.Yan, J. A.Shafer and J. P.Vacca. 1998. Efficacious, orally bioavailable thrombin inhibitors based on 3-aminopyridinone or 3-aminopyrazinone acetamide peptidomimetic templates. J Med Chem 41:4466-4474.

41. Gariepy,J. and K.Kawamura. 2001. Vectorial delivery of macromolecules into cells using peptide-based vehicles. Trends Biotechnol 19:21-28.

42. Janes,K. A., P.Calvo and M. J.Alonso. 2001. Polysaccharide colloidal particles as delivery systems for macromolecules. Adv Drug Deliv Rev 47:83-97.

43. Kreuter,J. 2001. Nanoparticulate systems for brain delivery of drugs. Adv Drug Deliv Rev 47:65-81.

44. Kates,S. A., S. B.Daniels and F.Albericio. 1993. Automated allyl cleavage for continuous-flow synthesis of cyclic and branched peptides. Anal Biochem 212:303-310.

45. Strydom,D. J. and S. A.Cohen. 1994. Comparison of amino acid analyses by phenylisothiocyanate and 6-aminoquinolyl-N-hydroxysuccinimidyl carbamate precolumn derivatization. Anal Biochem 222:19-28.

46. Cohen,S. A. and D. P.Michaud. 1993. Synthesis of a fluorescent derivatizing reagent, 6-aminoquinolyl-N-hydroxysuccinimidyl carbamate, and its application for the analysis of hydrolysate amino acids via high-performance liquid chromatography. Anal Biochem 211:279-287.

47. Kissinger,C. R., H. E.Parge, D. R.Knighton, C. T.Lewis, L. A.Pelletier, A.Tempczyk, V. J.Kalish, K. D.Tucker, R. E.Showalter and E. W.Moomaw. 1995. Crystal structures of human calcineurin and the human FKBP12-FK506-calcineurin complex. Nature 378:641-644.

48. Spinella,M. J., A. B.Malik, J.Everitt and T. T.Andersen. 1991. Design and synthesis of a specific endothelin 1 antagonist: effects on pulmonary vasoconstriction. Proc Natl Acad Sci USA 88:7443-7446.

49. Vives,E., P.Brodin and B.Lebleu. 1997. A truncated HIV-1 Tat protein basic domain rapidly translocates through the plasma membrane and accumulates in the cell nucleus. J Biol Chem 272:16010-16017.

50. Bonny,C., A.Oberson, S.Negri, C.Sauser and D. F.Schorderet. 2001. Cell-permeable peptide inhibitors of JNK: novel blockers of beta-cell death. Diabetes 50:77-82.

51. Nagahara,H., A. M.Vocero-Akbani, E. L.Snyder, A.Ho, D. G.Latham, N. A.Lissy, M.Becker-Hapak, S. A.Ezhevsky and S. F.Dowdy. 1998. Transduction of full-length TAT fusion proteins into mammalian cells: TAT-p27Kip1 induces cell migration. Nat Med 4:1449-1452.

52. Sambrook, J., Fritsch, E. F., and Maniatis, T. (1989) in Molecular Cloning: A laboratory Manual, Cold Spring Harbor Laboratory Press, Cold Spring Harbor, N.Y.

53. Crawford, D. R., Edbauer-Nechamen, C. A., Lowry, C. V., Salmon, S. L., Kim, Y. K., Davies, J. M. S., and Davies, K. J. A. (1994) Methods in Enzymology 234, 175-217

54. Laemmli, U. K. (1970) Nature 227, 680-685

55. Zhang, Y., Marcillat, O., Giulivi, C., Ernster, L., and Davies, K. J. (1990) J. Biol. Chem. 265, 16330-16336

56. Davies, K. J., Lloyd, D., and Boddy, L. (1989) J. Gen. Microbiol. 135, 2445-2451

57. Wiese, A. G., Pacifici, R. E., and Davies, K. J. A. (1995) Arch. Biochem. Biophys 318, 231-240

58. Crawford, D. R., Schools, G. P., Salmon, S. L., and Davies, K. J. A. (1996) Arch. Biochem. Biophys. 325, 256-264

59. Crawford, D. R., Schools, G. P., and Davies, K. J. A. (1996) Arch. Biochem. Biophys. 329, 137-144

60. Morris, J. A., Dorner, A. J., Edwards, C. A., Hendershot, L. M., and Kaufman, R. J. (1997) J. Biol. Chem. 272, 4327-4334

61. McCormick, T. S., McColl, K. S., and Distelhorst, C. W. (1997) J. Biol. Chem. 272, 6087-6092

62. Abramova, N. E., Davies, K. J. A., and Crawford, D. R. (2000) Free Radic. Biol. Med., in press.

63. Crawford, D. R., Leahy, K. P., Abramova, N., Lan, L., Wang, Y., and Davies, K. J. A. (1997) Arch. Biochem. Biophys. 342, 6-12.

64. Wooden, S. K., Kapur, R. P., and Lee, A. S. (1988) Exp. Cell Res. 178, 84-92

What is claimed is:

1. An isolated peptide having the formula
X₁KQFLISPPASPPVX₂ (SEQ ID NO:1)
where X₁ and X₂, together, contain 0 to 50 amino acids, and said peptide has calcineurin modulating activity.

2. The peptide of claim 1, where X₁ and X₂, together contain 0 to 34 amino acids.

3. The peptide of claim 1, wherein one or more of the S residues are phosphorylated.

4. An isolated peptide having the formula
X₁Xaa₁Xaa₂FLISXaa₃Xaa₄AS Xaa₅Xaa₆VX₂ (SEQ ID NO:7)
where
X₁ and X₂, together, contain 0 to 50 amino acids;
Xaa₁ is lysine or arginine;
Xaa₂ is glutamine, asparagine or glutamate;
Xaa₃, Xaa₄, Xaa₅, or Xaa₆ is proline or hydroxyproline; and
said peptide has calcineurin modulating activity.

5. The peptide of claim 4, where X₁ and X₂, together contain 0 to 34 amino acids.

6. The peptide of claim 4, wherein one or more of the S residues are phosphorylated.

7. An isolated active Adapt78 peptide fragment having the formula
X₁KQFLISPPASPPVX₂ (SEQ ID NO:1)
where X₁ and X₂, together, contain 0 to 50 amino acids, and said peptide has calcineurin modulating activity.

8. An isolated active Adapt78 peptide having the formula
X₁KQFLISPPASPPVX₂ (SEQ ID NO:1)
where X₁ and X₂, together, contain 0 to 34 amino acids, and said peptide has calcineurin modulating activity.

9. An isolated peptide having the formula
KQFLISPPASPPV (SEQ ID NO:2)
where said peptide has calcineurin modulating activity.

10. An isolated peptide having the formula
GRKKRRQRRR-GG (SEQ ID NO:5)
where said peptide has calcineurin modulating activity.

11. An isolated peptide having the formula
EMERMPKP (SEQ ID NO:6)
where said peptide has calcineurin modulating activity.

12. An isolated peptide having the formula
PDKQFLISPPASPPVGWKQVPKPKIIQTRRPE (SEQ ID NO:8)
where said peptide has calcineurin modulating activity.

13. An isolated fusion protein comprising SEQ ID NO: 2 and SEQ ID NO: 3.

14. The fusion protein of claim 13, having the sequence of SEQ ID NO: 4.

15. A transgenic animal that overexpresses a human Adapt78 protein or human Adapt78 polypeptide.

16. The transgenic animal of claim 15, wherein the human Adapt78 protein or human Adapt78 polypeptide that is expressed is the peptide of claim 1.

17. The transgenic animal of claim 15, wherein the human Adapt78 protein or human Adapt78 polypeptide that is expressed is the peptide of claim 4.

18. The transgenic animal of claim 15, wherein the human Adapt78 protein or human Adapt78 polypeptide that is expressed is the peptide of claim 7.

19. The transgenic animal of claim 15, wherein the human Adapt78 protein or human Adapt78 polypeptide that is expressed is the peptide of claim 8.

20. An antibody specific for an Adapt78 protein.

21. An antibody specific for an Adapt78 polypeptide.

22. The antibody of claim 21, which is a polyclonal antibody raised against the peptide of SEQ ID NO: 6.

23. A host cell genetically engineered to overexpress a human Adapt78 protein or human Adapt78 polypeptide.

24. An isolated polypeptide comprising an amino acid sequence which is selected from the group consisting of SEQ ID NOs: 1, 2, 4, 5, 6, 7 and 8, the translated protein coding portion thereof, the mature protein coding portion thereof or the extracellular portion thereof.

25. A composition comprising the polypeptide of claim 24 and a pharmaceutically acceptable carrier.

26. A method of modulating calcineurin activity in a subject, comprising administering a polypeptide comprising an amino acid sequence which is selected from the group consisting of SEQ ID NOs: 1, 2, 4, 5, 6, 7 and 8, to a subject, such that calcineurin activity is modulated.

27. The method of claim 26, wherein the peptide is the peptide of SEQ ID NO: 1.

28. The method of claim 26, wherein the peptide is the peptide of SEQ ID NO: 2.

29. The method of claim 26, wherein the peptide is the peptide of SEQ ID NO: 4.

30. The method of claim 26, wherein the peptide is the peptide of SEQ ID NO: 5.

31. The method of claim 26, wherein the peptide is the peptide of SEQ ID NO: 6.

32. The method of claim 26, wherein the peptide is the peptide of SEQ ID NO: 7.

33. The method of claim 26, wherein the peptide is the peptide of SEQ ID NO: 8.

34. The method of claim 26, wherein said calcineurin activity is down regulated.

35. A method of treating a condition characterized by calcineurin overexpression or overactivation, comprising administering a polypeptide comprising an amino acid sequence which is selected from the group consisting of SEQ ID NOs: 1, 2, 4, 5, 6, 7 and 8 to a subject, such that calcineurin activity is modulated and said condition is treated.

36. The method of claim 35, wherein the peptide is the peptide of SEQ ID NO: 1.

37. The method of claim 35, wherein the peptide is the peptide of SEQ ID NO: 2.

38. The method of claim 35, wherein the peptide is the peptide of SEQ ID NO: 4.

39. The method of claim 35, wherein the peptide is the peptide of SEQ ID NO: 5.

40. The method of claim 35, wherein the peptide is the peptide of SEQ ID NO: 6.

41. The method of claim 35, wherein the peptide is the peptide of SEQ ID NO: 7.

42. The method of claim 35, wherein the peptide is the peptide of SEQ ID NO: 8.

43. The method of claim 35, wherein said condition is selected from the group consisting of cancer, immune system and brain disorders, skeletal and cardiac muscle dysfunction, cardiac hypertrophy and heart failure.

44. The method of claim 35, wherein said administration is accomplished without substantial adverse side effects.

45. The method of claim 35, wherein said peptide is administered in conjunction with another calcineurin inhibitor.

46. The method of claim 45, wherein said another calcineurin inhibitor is selected from the group consisting of cyclosporin A and FK506.

47. A method for determining candidate modulators of Adapt78 proteins or Adapt78 polypeptides, comprising contacting a candidate modulator and an Adapt78 protein or Adapt78 polypeptide to a cell sample in which calcineurin is active, and determining the effect of said candidate modulator compared to the effect of said Adapt78 protein or Adapt78 polypeptide on said cell sample in the absence of said candidate modulator.

48. The method of claim 47, wherein said cell sample comprises cells selected from the group consisting of IMR-90 fibroblasts; U251 astroglioma cells; MCF7 breast adenocarcinoma cells; HeLa epitheliod carcinoma cells; HL60 promyclocytes; BEC(2)-M17 neuroblastoma cells; and primary mouse cardiomyocyte cells.

49. The method of claim 48, wherein said cells are human cells.

50. A method of treating Alzheimer's Disease, comprising administering, to a patient in need thereof, an effective amount of a peptide of claim 1 to a subject, such that Alzheimer's Disease is treated in said patient.

51. A method of treating cancer, comprising administering, to a patient in need thereof, an effective amount of a peptide of claim 1 to a subject, such that said cancer is treated in said patient.

52. A method of treating cardiac hypertrophy, comprising administering, to a patient in need thereof, an effective amount of a peptide of claim 1 to a subject, such that cardiac hypertrophy is treated in said patient.

53. A method of treating immune system dysfunction, comprising administering, to a patient in need thereof, an effective amount of a peptide of claim 1 to a subject, such that immune system dysfunction is treated in said patient.
